SEQUENCE LISTING (175 sequences)

SEQ ID NO: 1 - protein, 999 aa, Homo sapiens
  human MERTK (including signal sequence) (UniProt KB Q12866)
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 2 - protein, 12 aa, Homo sapiens
  identified peptide of rhMER_Fc after trypsin proteolysis
HTVGHIILSEHK

SEQ ID NO: 3 - protein, 15 aa, Homo sapiens
  identified peptide of rhMER_Fc after trypsin proteolysis
HTVGHIILSEHKGVK

SEQ ID NO: 4 - protein, 23 aa, Homo sapiens
  identified peptide of rhMER_Fc after trypsin proteolysis
FNCSISVPNIYQDTTISWWKDGK

SEQ ID NO: 5 - protein, 9 aa, Artificial Sequence
  identified peptide of rhMER_Fc after trypsin proteolysis
  misc_feature (8)..(8): Xaa is Carbamidomethyl
SDNGSYIXK

SEQ ID NO: 6 - protein, 25 aa, Artificial Sequence
  identified peptide of rhMER_Fc after trypsin proteolysis
  misc_feature (1)..(1): Xaa is Met-Oxidation
XKINNEEIVSDPIYIEVQGLPHFTK

SEQ ID NO: 7 - protein, 23 aa, Homo sapiens
  identified peptide of rhMER_Fc after trypsin proteolysis
INNEEIVSDPIYIEVQGLPHFTK

SEQ ID NO: 8 - protein, 9 aa, Homo sapiens
  identified peptide of rhMER_Fc after trypsin proteolysis
QPESMNVTR

SEQ ID NO: 9 - protein, 37 aa, Homo sapiens
  identified peptide of rhMER_Fc after trypsin proteolysis
SPSVLTVPGLTEMAVFSCEAHNDKGLTVSKGVQINIK

SEQ ID NO: 10 - protein, 12 aa, Homo sapiens
  identified peptide of rhMER_Fc after trypsin proteolysis
AIPSPPTEVSIR

SEQ ID NO: 11 - protein, 22 aa, Homo sapiens
  identified peptide of rhMER_Fc after trypsin proteolysis
NSTAHSILISWVPGFDGYSPFR

SEQ ID NO: 12 - protein, 7 aa, Artificial Sequence
  identified peptide of rhMER_Fc after trypsin proteolysis
  misc_feature (2)..(2): Xaa is Carbamidomethyl
NXSIQVK

SEQ ID NO: 13 - protein, 25 aa, Artificial Sequence
  identified peptide of rhMER_Fc after trypsin proteolysis
EADPLSNGSVMIFNTSALPHLYQIK

SEQ ID NO: 14 - protein, 7 aa, Artificial Sequence
  identified peptide of rhMER_Fc after trypsin proteolysis
  misc_feature (2)..(2): Xaa is Met-Oxidation
WXKPPTK

SEQ ID NO: 15 - protein, 17 aa, Artificial Sequence
  identified peptide of rhMER_Fc after trypsin proteolysis
  misc_feature (2)..(2): Xaa is Met-Oxidation
WXKPPTKQQDGELVGYR

SEQ ID NO: 16 - protein, 10 aa, Homo sapiens
  identified peptide of rhMER_Fc after trypsin proteolysis
QQDGELVGYR

SEQ ID NO: 17 - protein, 12 aa, Homo sapiens
  identified peptide of rhMER_Fc after trypsin proteolysis
ISHVWQSAGISK

SEQ ID NO: 18 - protein, 12 aa, Homo sapiens
  identified peptide of rhMER_Fc after trypsin proteolysis
ELLEEVGQNGSR

SEQ ID NO: 19 - protein, 15 aa, Artificial Sequence
  identified peptide of rhMER_Fc after trypsin proteolysis
  misc_feature (12)..(12): Xaa is Carbamidomethyl
ARISVQVHNATXTVR

SEQ ID NO: 20 - protein, 13 aa, Artificial Sequence
  identified peptide of rhMER_Fc after trypsin proteolysis
  misc_feature (10)..(10): Xaa is Carbamidomethyl
ISVQVHNATXTVR

SEQ ID NO: 21 - protein, 17 aa, Homo sapiens
  identified peptide of rhMER_Fc after trypsin proteolysis
IAAVTRGGVGPFSDPVK

SEQ ID NO: 22 - protein, 11 aa, Homo sapiens
  identified peptide of rhMER_Fc after trypsin proteolysis
GGVGPFSDPVK

SEQ ID NO: 23 - protein, 24 aa, Homo sapiens
  identified peptide of rhMER_Fc after Chymotrypsin proteolysis
SPFRNCSIQVKEADPLSNGSVMIF

SEQ ID NO: 24 - protein, 20 aa, Homo sapiens
  identified peptide of rhMER_Fc after ASP-N proteolysis
REEAKPYPLFPGPFPGSLQT

SEQ ID NO: 25 - protein, 12 aa, Artificial Sequence
  identified peptide of rhMER_Fc after ASP-N proteolysis
  misc_feature (5)..(5): Xaa is Met-Oxidation
DIRWXKPPTKQQ

SEQ ID NO: 26 - protein, 13 aa, Homo sapiens
  identified peptide of rhMER_Fc after ASP-N proteolysis
DPVKIFIPAHGWV

SEQ ID NO: 27 - protein, 13 aa, Homo sapiens
  identified peptide of rhMER_Fc after ASP-N proteolysis
DYAPSSTPAPGNA

SEQ ID NO: 28 - protein, 9 aa, Homo sapiens
  identified peptide of rhMER_Fc after Elastase proteolysis
REEAKPYPL

SEQ ID NO: 29 - protein, 18 aa, Homo sapiens
  identified peptide of rhMER_Fc after Elastase proteolysis
REEAKPYPLFPGPFPGSL

SEQ ID NO: 30 - protein, 16 aa, Homo sapiens
  identified peptide of rhMER_Fc after Elastase proteolysis
FPGPFPGSLQTDHTPL

SEQ ID NO: 31 - protein, 5 aa, Homo sapiens
  identified peptide of rhMER_Fc after Elastase proteolysis
GYQPA

SEQ ID NO: 32 - protein, 16 aa, Artificial Sequence
  identified peptide of rhMER_Fc after Elastase proteolysis
  misc_feature (2)..(2): Xaa is Met-Oxidation
LXFSPTQPGRPHTGNV

SEQ ID NO: 33 - protein, 17 aa, Homo sapiens
  identified peptide of rhMER_Fc after Elastase proteolysis
LMFSPTQPGRPHTGNVA

SEQ ID NO: 34 - protein, 7 aa, Homo sapiens
  identified peptide of rhMER_Fc after Elastase proteolysis
IPQVTSV

SEQ ID NO: 35 - protein, 9 aa, Homo sapiens
  identified peptide of rhMER_Fc after Elastase proteolysis
ESKPLPPLA

SEQ ID NO: 36 - protein, 14 aa, Homo sapiens
  identified peptide of rhMER_Fc after Elastase proteolysis
ESKPLPPLAFKHTV

SEQ ID NO: 37 - protein, 9 aa, Homo sapiens
  identified peptide of rhMER_Fc after Elastase proteolysis
FKHTVGHII

SEQ ID NO: 38 - protein, 8 aa, Homo sapiens
  identified peptide of rhMER_Fc after Elastase proteolysis
ILSEHKGV

SEQ ID NO: 39 - protein, 7 aa, Homo sapiens
  identified peptide of rhMER_Fc after Elastase proteolysis
KFNCSIS

SEQ ID NO: 40 - protein, 10 aa, Homo sapiens
  identified peptide of rhMER_Fc after Elastase proteolysis
VPNIYQDTTI

SEQ ID NO: 41 - protein, 9 aa, Homo sapiens
  identified peptide of rhMER_Fc after Elastase proteolysis
SWWKDGKEL

SEQ ID NO: 42 - protein, 5 aa, Homo sapiens
  identified peptide of rhMER_Fc after Elastase proteolysis
GAHHA

SEQ ID NO: 43 - protein, 12 aa, Homo sapiens
  identified peptide of rhMER_Fc after Elastase proteolysis
ITQFYPDDEVTA

SEQ ID NO: 44 - protein, 12 aa, Homo sapiens
  identified peptide of rhMER_Fc after Elastase proteolysis
TQFYPDDEVTAI

SEQ ID NO: 45 - protein, 9 aa, Homo sapiens
  identified peptide of rhMER_Fc after Elastase proteolysis
QRSDNGSYI

SEQ ID NO: 46 - protein, 11 aa, Homo sapiens
  identified peptide of rhMER_Fc after Elastase proteolysis
CKMKINNEEIV

SEQ ID NO: 47 - protein, 10 aa, Homo sapiens
  identified peptide of rhMER_Fc after Elastase proteolysis
NNEEIVSDPI

SEQ ID NO: 48 - protein, 7 aa, Homo sapiens
  identified peptide of rhMER_Fc after Elastase proteolysis
DPIYIEV

SEQ ID NO: 49 - protein, 15 aa, Homo sapiens
  identified peptide of rhMER_Fc after Elastase proteolysis
QGLPHFTKQPESMNV

SEQ ID NO: 50 - protein, 12 aa, Homo sapiens
  identified peptide of rhMER_Fc after Elastase proteolysis
TRNTAFNLTCQA

SEQ ID NO: 51 - protein, 11 aa, Homo sapiens
  identified peptide of rhMER_Fc after Elastase proteolysis
TCQAVGPPEPV

SEQ ID NO: 52 - protein, 12 aa, Homo sapiens
  identified peptide of rhMER_Fc after Elastase proteolysis
SRVNEQPEKSPS

SEQ ID NO: 53 - protein, 11 aa, Homo sapiens
  identified peptide of rhMER_Fc after Elastase proteolysis
NEQPEKSPSVL

SEQ ID NO: 54 - protein, 10 aa, Homo sapiens
  identified peptide of rhMER_Fc after Elastase proteolysis
TVPGLTEMAV

SEQ ID NO: 55 - protein, 12 aa, Artificial Sequence
  identified peptide of rhMER_Fc after Elastase proteolysis
  misc_feature (4)..(4): Xaa is Carbamidomethyl
VFSXEAHNDKGL

SEQ ID NO: 56 - protein, 11 aa, Artificial Sequence
  identified peptide of rhMER_Fc after Elastase proteolysis
  misc_feature (3)..(3): Xaa is Carbamidomethyl
FSXEAHNDKGL

SEQ ID NO: 57 - protein, 13 aa, Artificial Sequence
  identified peptide of rhMER_Fc after Elastase proteolysis
  misc_feature (3)..(3): Xaa is Carbamidomethyl
FSXEAHNDKGLTV

SEQ ID NO: 58 - protein, 8 aa, Homo sapiens
  identified peptide of rhMER_Fc after Elastase proteolysis
HNDKGLTV

SEQ ID NO: 59 - protein, 6 aa, Homo sapiens
  identified peptide of rhMER_Fc after Elastase proteolysis
QINIKA

SEQ ID NO: 60 - protein, 12 aa, Homo sapiens
  identified peptide of rhMER_Fc after Elastase proteolysis
NIKAIPSPPTEV

SEQ ID NO: 61 - protein, 10 aa, Homo sapiens
  identified peptide of rhMER_Fc after Elastase proteolysis
SIRNSTAHSI

SEQ ID NO: 62 - protein, 5 aa, Homo sapiens
  identified peptide of rhMER_Fc after Elastase proteolysis
SWVPG

SEQ ID NO: 63 - protein, 11 aa, Homo sapiens
  identified peptide of rhMER_Fc after Elastase proteolysis
FDGYSPFRNCS

SEQ ID NO: 64 - protein, 11 aa, Homo sapiens
  identified peptide of rhMER_Fc after Elastase proteolysis
YSPFRNCSIQV

SEQ ID NO: 65 - protein, 5 aa, Homo sapiens
  identified peptide of rhMER_Fc after Elastase proteolysis
QVKEA

SEQ ID NO: 66 - protein, 9 aa, Homo sapiens
  identified peptide of rhMER_Fc after Elastase proteolysis
FNTSALPHL

SEQ ID NO: 67 - protein, 7 aa, Homo sapiens
  identified peptide of rhMER_Fc after Elastase proteolysis
LPHLYQI

SEQ ID NO: 68 - protein, 10 aa, Homo sapiens
  identified peptide of rhMER_Fc after Elastase proteolysis
LPHLYQIKQL

SEQ ID NO: 69 - protein, 6 aa, Homo sapiens
  identified peptide of rhMER_Fc after Elastase proteolysis
KQLQAL

SEQ ID NO: 70 - protein, 5 aa, Homo sapiens
  identified peptide of rhMER_Fc after Elastase proteolysis
ANYSI

SEQ ID NO: 71 - protein, 7 aa, Homo sapiens
  identified peptide of rhMER_Fc after Elastase proteolysis
VSCMNEI

SEQ ID NO: 72 - protein, 10 aa, Artificial Sequence
  identified peptide of rhMER_Fc after Elastase proteolysis
  misc_feature (2)..(2): Xaa is Carbamidomethyl
SXMNEIGWSA

SEQ ID NO: 73 - protein, 6 aa, Homo sapiens
  identified peptide of rhMER_Fc after Elastase proteolysis
SPWILA

SEQ ID NO: 74 - protein, 8 aa, Homo sapiens
  identified peptide of rhMER_Fc after Elastase proteolysis
TTEGAPSV

SEQ ID NO: 75 - protein, 7 aa, Homo sapiens
  identified peptide of rhMER_Fc after Elastase proteolysis
APLNVTV

SEQ ID NO: 76 - protein, 7 aa, Homo sapiens
  identified peptide of rhMER_Fc after Elastase proteolysis
TVFLNES

SEQ ID NO: 77 - protein, 7 aa, Homo sapiens
  identified peptide of rhMER_Fc after Elastase proteolysis
NESSDNV

SEQ ID NO: 78 - protein, 17 aa, Homo sapiens
  identified peptide of rhMER_Fc after Elastase proteolysis
DIRWMKPPTKQQDGELV

SEQ ID NO: 79 - protein, 14 aa, Homo sapiens
  identified peptide of rhMER_Fc after Elastase proteolysis
RWMKPPTKQQDGEL

SEQ ID NO: 80 - protein, 15 aa, Artificial Sequence
  identified peptide of rhMER_Fc after Elastase proteolysis
  misc_feature (3)..(3): Xaa is Met-Oxidation
RWXKPPTKQQDGELV

SEQ ID NO: 81 - protein, 19 aa, Homo sapiens
  identified peptide of rhMER_Fc after Elastase proteolysis
RWMKPPTKQQDGELVGYRI

SEQ ID NO: 82 - protein, 6 aa, Homo sapiens
  identified peptide of rhMER_Fc after Elastase proteolysis
YRISHV

SEQ ID NO: 83 - protein, 5 aa, Homo sapiens
  identified peptide of rhMER_Fc after Elastase proteolysis
WQSAG

SEQ ID NO: 84 - protein, 5 aa, Homo sapiens
  identified peptide of rhMER_Fc after Elastase proteolysis
ISKEL

SEQ ID NO: 85 - protein, 8 aa, Homo sapiens
  identified peptide of rhMER_Fc after Elastase proteolysis
SKELLEEV

SEQ ID NO: 86 - protein, 5 aa, Homo sapiens
  identified peptide of rhMER_Fc after Elastase proteolysis
SRARI

SEQ ID NO: 87 - protein, 5 aa, Homo sapiens
  identified peptide of rhMER_Fc after Elastase proteolysis
RARIS

SEQ ID NO: 88 - protein, 9 aa, Homo sapiens
  identified peptide of rhMER_Fc after Elastase proteolysis
QVHNATCTV

SEQ ID NO: 89 - protein, 9 aa, Homo sapiens
  identified peptide of rhMER_Fc after Elastase proteolysis
HNATCTVRI

SEQ ID NO: 90 - protein, 13 aa, Homo sapiens
  identified peptide of rhMER_Fc after Elastase proteolysis
VTRGGVGPFSDPV

SEQ ID NO: 91 - protein, 12 aa, Homo sapiens
  identified peptide of rhMER_Fc after Elastase proteolysis
TRGGVGPFSDPV

SEQ ID NO: 92 - protein, 9 aa, Homo sapiens
  identified peptide of rhMER_Fc after Elastase proteolysis
DPVKIFIPA

SEQ ID NO: 93 - protein, 9 aa, Homo sapiens
  identified peptide of rhMER_Fc after Elastase proteolysis
HGWVDYAPS

SEQ ID NO: 94 - protein, 11 aa, Homo sapiens
  identified peptide of rhMER_Fc after Elastase proteolysis
DYAPSSTPAPG

SEQ ID NO: 95 - protein, 8 aa, Homo sapiens
  identified peptide of rhMER_Fc after Thermolysin proteolysis
REEAKPYP

SEQ ID NO: 96 - protein, 14 aa, Homo sapiens
  identified peptide of rhMER_Fc after Thermolysin proteolysis
AKPYPLFPGPFPGS

SEQ ID NO: 97 - protein, 17 aa, Homo sapiens
  identified peptide of rhMER_Fc after Thermolysin proteolysis
LFPGPFPGSLQTDHTPL

SEQ ID NO: 98 - protein, 12 aa, Homo sapiens
  identified peptide of rhMER_Fc after Thermolysin proteolysis
LSLPHASGYQPA

SEQ ID NO: 99 - protein, 15 aa, Homo sapiens
  identified peptide of rhMER_Fc after Thermolysin proteolysis
LMFSPTQPGRPHTGN

SEQ ID NO: 100 - protein, 14 aa, Homo sapiens
  identified peptide of rhMER_Fc after Thermolysin proteolysis
MFSPTQPGRPHTGN

SEQ ID NO: 101 - protein, 13 aa, Homo sapiens
  identified peptide of rhMER_Fc after Thermolysin proteolysis
FSPTQPGRPHTGN

SEQ ID NO: 102 - protein, 18 aa, Homo sapiens
  identified peptide of rhMER_Fc after Thermolysin proteolysis
FSPTQPGRPHTGNVAIPQ

SEQ ID NO: 103 - protein, 11 aa, Homo sapiens
  identified peptide of rhMER_Fc after Thermolysin proteolysis
VTSVESKPLPP

SEQ ID NO: 104 - protein, 8 aa, Homo sapiens
  identified peptide of rhMER_Fc after Thermolysin proteolysis
VESKPLPP

SEQ ID NO: 105 - protein, 8 aa, Homo sapiens
  identified peptide of rhMER_Fc after Thermolysin proteolysis
AFKHTVGH

SEQ ID NO: 106 - protein, 8 aa, Homo sapiens
  identified peptide of rhMER_Fc after Thermolysin proteolysis
IILSEHKG

SEQ ID NO: 107 - protein, 12 aa, Homo sapiens
  identified peptide of rhMER_Fc after Thermolysin proteolysis
LSEHKGVKFNCS

SEQ ID NO: 108 - protein, 11 aa, Homo sapiens
  identified peptide of rhMER_Fc after Thermolysin proteolysis
VKFNCSISVPN

SEQ ID NO: 109 - protein, 15 aa, Homo sapiens
  identified peptide of rhMER_Fc after Thermolysin proteolysis
IYQDTTISWWKDGKE

SEQ ID NO: 110 - protein, 16 aa, Homo sapiens
  identified peptide of rhMER_Fc after Thermolysin proteolysis
IYQDTTISWWKDGKEL

SEQ ID NO: 111 - protein, 9 aa, Homo sapiens
  identified peptide of rhMER_Fc after Thermolysin proteolysis
ISWWKDGKE

SEQ ID NO: 112 - protein, 10 aa, Homo sapiens
  identified peptide of rhMER_Fc after Thermolysin proteolysis
AITQFYPDDE

SEQ ID NO: 113 - protein, 11 aa, Homo sapiens
  identified peptide of rhMER_Fc after Thermolysin proteolysis
ITQFYPDDEVT

SEQ ID NO: 114 - protein, 14 aa, Homo sapiens
  identified peptide of rhMER_Fc after Thermolysin proteolysis
FSITSVQRSDNGSY

SEQ ID NO: 115 - protein, 10 aa, Artificial Sequence
  identified peptide of rhMER_Fc after Thermolysin proteolysis
  misc_feature (2)..(2): Xaa is Carbamidomethyl
IXKMKINNEE

SEQ ID NO: 116 - protein, 7 aa, Homo sapiens
  identified peptide of rhMER_Fc after Thermolysin proteolysis
IVSDPIY

SEQ ID NO: 117 - protein, 15 aa, Homo sapiens
  identified peptide of rhMER_Fc after Thermolysin proteolysis
IEVQGLPHFTKQPES

SEQ ID NO: 118 - protein, 9 aa, Homo sapiens
  identified peptide of rhMER_Fc after Thermolysin proteolysis
FTKQPESMN

SEQ ID NO: 119 - protein, 8 aa, Homo sapiens
  identified peptide of rhMER_Fc after Thermolysin proteolysis
MNVTRNTA

SEQ ID NO: 120 - protein, 7 aa, Homo sapiens
  identified peptide of rhMER_Fc after Thermolysin proteolysis
FNLTCQA

SEQ ID NO: 121 - protein, 9 aa, Homo sapiens
  identified peptide of rhMER_Fc after Thermolysin proteolysis
IFWVQNSSR

SEQ ID NO: 122 - protein, 16 aa, Homo sapiens
  identified peptide of rhMER_Fc after Thermolysin proteolysis
VNEQPEKSPSVLTVPG

SEQ ID NO: 123 - protein, 9 aa, Homo sapiens
  identified peptide of rhMER_Fc after Thermolysin proteolysis
LTVPGLTEM

SEQ ID NO: 124 - protein, 11 aa, Artificial Sequence
  identified peptide of rhMER_Fc after Thermolysin proteolysis
  misc_feature (4)..(4): Xaa is Carbamidomethyl
VFSXEAHNDKG

SEQ ID NO: 125 - protein, 10 aa, Artificial Sequence
  identified peptide of rhMER_Fc after Thermolysin proteolysis
  misc_feature (3)..(3): Xaa is Carbamidomethyl
FSXEAHNDKG

SEQ ID NO: 126 - protein, 8 aa, Homo sapiens
  identified peptide of rhMER_Fc after Thermolysin proteolysis
LTVSKGVQ

SEQ ID NO: 127 - protein, 12 aa, Homo sapiens
  identified peptide of rhMER_Fc after Thermolysin proteolysis
INIKAIPSPPTE

SEQ ID NO: 128 - protein, 12 aa, Homo sapiens
  identified peptide of rhMER_Fc after Thermolysin proteolysis
IKAIPSPPTEVS

SEQ ID NO: 129 - protein, 15 aa, Homo sapiens
  identified peptide of rhMER_Fc after Thermolysin proteolysis
AIPSPPTEVSIRNST

SEQ ID NO: 130 - protein, 8 aa, Homo sapiens
  identified peptide of rhMER_Fc after Thermolysin proteolysis
IRNSTAHS

SEQ ID NO: 131 - protein, 8 aa, Homo sapiens
  identified peptide of rhMER_Fc after Thermolysin proteolysis
ILISWVPG

SEQ ID NO: 132 - protein, 13 aa, Homo sapiens
  identified peptide of rhMER_Fc after Thermolysin proteolysis
LISWVPGFDGYSP

SEQ ID NO: 133 - protein, 13 aa, Homo sapiens
  identified peptide of rhMER_Fc after Thermolysin proteolysis
FDGYSPFRNCSIQ

SEQ ID NO: 134 - protein, 10 aa, Homo sapiens
  identified peptide of rhMER_Fc after Thermolysin proteolysis
ALPHLYQIKQ

SEQ ID NO: 135 - protein, 8 aa, Homo sapiens
  identified peptide of rhMER_Fc after Thermolysin proteolysis
LYQIKQLQ

SEQ ID NO: 136 - protein, 6 aa, Homo sapiens
  identified peptide of rhMER_Fc after Thermolysin proteolysis
IKQLQA

SEQ ID NO: 137 - protein, 7 aa, Homo sapiens
  identified peptide of rhMER_Fc after Thermolysin proteolysis
LANYSIG

SEQ ID NO: 138 - protein, 6 aa, Homo sapiens
  identified peptide of rhMER_Fc after Thermolysin proteolysis
VSCMNE

SEQ ID NO: 139 - protein, 10 aa, Homo sapiens
  identified peptide of rhMER_Fc after Thermolysin proteolysis
LASTTEGAPS

SEQ ID NO: 140 - protein, 5 aa, Homo sapiens
  identified peptide of rhMER_Fc after Thermolysin proteolysisTBA
LNVTV

SEQ ID NO: 141 - protein, 16 aa, Homo sapiens
  identified peptide of rhMER_Fc after Thermolysin proteolysis
VDIRWMKPPTKQQDGE

SEQ ID NO: 142 - protein, 11 aa, Homo sapiens
  identified peptide of rhMER_Fc after Thermolysin proteolysis
MKPPTKQQDGE

SEQ ID NO: 143 - protein, 12 aa, Homo sapiens
  identified peptide of rhMER_Fc after Thermolysin proteolysis
MKPPTKQQDGEL

SEQ ID NO: 144 - protein, 16 aa, Artificial Sequence
  identified peptide of rhMER_Fc after Thermolysin proteolysis
  misc_feature (1)..(1): Xaa is Met-Oxidation
XKPPTKQQDGELVGYR

SEQ ID NO: 145 - protein, 8 aa, Homo sapiens
  identified peptide of rhMER_Fc after Thermolysin proteolysis
LVGYRISH

SEQ ID NO: 146 - protein, 7 aa, Homo sapiens
  identified peptide of rhMER_Fc after Thermolysin proteolysis
VGYRISH

SEQ ID NO: 147 - protein, 9 aa, Homo sapiens
  identified peptide of rhMER_Fc after Thermolysin proteolysis
ISHVWQSAG

SEQ ID NO: 148 - protein, 5 aa, Homo sapiens
  identified peptide of rhMER_Fc after Thermolysin proteolysis
ISKEL

SEQ ID NO: 149 - protein, 12 aa, Homo sapiens
  identified peptide of rhMER_Fc after Thermolysin proteolysis
LEEVGQNGSRAR

SEQ ID NO: 150 - protein, 11 aa, Homo sapiens
  identified peptide of rhMER_Fc after Thermolysin proteolysis
VGQNGSRARIS

SEQ ID NO: 151 - protein, 5 aa, Homo sapiens
  identified peptide of rhMER_Fc after Thermolysin proteolysis
VQVHN

SEQ ID NO: 152 - protein, 6 aa, Homo sapiens
  identified peptide of rhMER_Fc after Thermolysin proteolysis
TCTVRI

SEQ ID NO: 153 - protein, 9 aa, Homo sapiens
  identified peptide of rhMER_Fc after Thermolysin proteolysis
AVTRGGVGP

SEQ ID NO: 154 - protein, 7 aa, Homo sapiens
  identified peptide of rhMER_Fc after Thermolysin proteolysis
FSDPVKI

SEQ ID NO: 155 - protein, 20 aa, Homo sapiens
  identified peptide of rhMER_Fc after Thermolysin proteolysis
FIPAHGWVDYAPSSTPAPGN

SEQ ID NO: 156 - protein, 13 aa, Artificial Sequence
  peptide of z10_VL
TWYQQKPGKAPKL

SEQ ID NO: 157 - protein, 5 aa, Artificial Sequence
  peptide of RhMer_Fc
STTEG

SEQ ID NO: 158 - protein, 16 aa, Artificial Sequence
  peptide of z10_VH
EWMGWINTYTGEPTYA

SEQ ID NO: 159 - protein, 16 aa, Artificial Sequence
  peptide of RhMer_Fc
EGAPSVAPLNVTVFLN

SEQ ID NO: 160 - protein, 11 aa, Artificial Sequence
  peptide of z10_VH
CARKSTVVSRY

SEQ ID NO: 161 - protein, 17 aa, Artificial Sequence
  peptide of RhMer_Fc
NVTVFLNESSDNVDIRW

SEQ ID NO: 162 - protein, 26 aa, Artificial Sequence
  peptide of z10_VL
DFATYYCQQYRSYPLTFGQGTKLEIK

SEQ ID NO: 163 - protein, 18 aa, Artificial Sequence
  peptide of RhMer_Fc
ESSDNVDIRWMKPPTKQQ

SEQ ID NO: 164 - protein, 9 aa, Artificial Sequence
  peptide of z10_VH
SGYTFTNYG

SEQ ID NO: 165 - protein, 17 aa, Artificial Sequence
  peptide of RhMer_Fc
DNVDIRWMKPPTKQQDG

SEQ ID NO: 166 - protein, 20 aa, Artificial Sequence
  peptide of z10_VH
RSDDMAVYYCARKSTVVSRY

SEQ ID NO: 167 - protein, 15 aa, Artificial Sequence
  peptide of RhMer_Fc
MKPPTKQQDGELVGY

SEQ ID NO: 168 - protein, 9 aa, Artificial Sequence
  peptide of z10_VL
CQQYRSYPL

SEQ ID NO: 169 - protein, 12 aa, Artificial Sequence
  peptide of RhMer_Fc
MKPPTKQQDGEL

SEQ ID NO: 170 - protein, 17 aa, Artificial Sequence
  peptide of z10_VH
INTYTGEPTYADDFKGR

SEQ ID NO: 171 - protein, 16 aa, Artificial Sequence
  peptide of RhMer_Fc
MKPPTKQQDGELVGYR

SEQ ID NO: 172 - protein, 16 aa, Artificial Sequence
  peptide of z10_VH
TNYGMNWVRQAPGQGL

SEQ ID NO: 173 - protein, 21 aa, Artificial Sequence
  peptide of RhMer_Fc
MKPPTKQQDGELVGYRISHVW

SEQ ID NO: 174 - protein, 22 aa, Artificial Sequence
  peptide of z10_VL
WASTRHTGVPDRFSGSGSGTDF

SEQ ID NO: 175 - protein, 16 aa, Artificial Sequence
  peptide of RhMer_Fc
RISHVWQSAGISKELL

(19) United States
(12) Patent Application Publication
(10) Pub. No.: US 2024/0279347 A1
(43) Pub. Date: Aug. 22, 2024

(54) MERTK PEPTIDES AND USES THEREOF

(71) Applicant: Inspirna, Inc., New York, NY (US)

(72) Inventors: Masoud Tavazoie, New York, NY (US); Isabel Kurth, New York, NY (US); Shugaku Takeda, Queens Village, NY (US); David M. Darst, New York, NY (US)

(73) Assignee: Inspirna, Inc., New York, NY (US)

(21) Appl. No.: 18/559,718

(22) PCT Filed: May 13, 2022
(86) PCT No.: PCT/US2022/029185

§ 371 Date: Nov. 8, 2023

Related U.S. Application Data
(60) Provisional application No. 63/189,036, filed on May 14, 2021.

(51) Int. Cl. C07K 16/28; C07K 14/435; C12N 9/12

(52) U.S. Cl. CPC C07K 16/2863; C07K 14/43504; C12N 9/12; C12Y 207/10001; C07K 2317/34; C07K 2319/30

(57) ABSTRACT

The present disclosure provides peptides comprising amino acid sequences of human MERTK and uses thereof for the production and screening of antibodies.

### CROSS-REFERENCE TO RELATED APPLICATIONS

This application claims the benefit of U.S. Provisional Patent Application No. 63/189,036, filed May 14, 2021, which is incorporated by reference herein in its entirety.

### REFERENCE TO SEQUENCE LISTING SUBMITTED ELECTRONICALLY

This application incorporates by reference a Sequence Listing submitted with this application as a text file entitled “13256-012-228_Sequence_Listing.txt” created on May 4, 2022 and having a size of 53,121 bytes.

### 1. FIELD

The present disclosure provides polypeptides comprising amino acid sequences of human MERTK and uses thereof for the production and screening of antibodies.

### 2. BACKGROUND

Mer Tyrosine Kinase (MERTK), also referred to as c-mer, MER, Proto-oncogene c-Mer, Receptor Tyrosine Kinase MerTK, Tyrosine-protein Kinase Mer, STK Kinase, RP38, or MGC133349, is a member of the TAM family of receptor tyrosine kinases, which also include AXL and TYRO3 kinases. MERTK transduces signals from the extracellular space via activation by binding of ligands, most notably Gas-6, a soluble protein. Gas-6 binding to MERTK induces autophosphorylation of MERTK on its intracellular domain, resulting in downstream signal activation (Cummings C T et al., (2013) Clin Cancer Res 19; 5275-5280; Verma A et al., (2011) Mol Cancer Ther 10: 1763-1773).

MERTK exists in both membrane bound and soluble forms. The extracellular domain can be cleaved to generate a soluble extracellular domain, which is hypothesized to act as a decoy receptor to negatively regulate MERTK receptor activation on cells by reducing the ability and/or availability of soluble Gas-6 ligand to bind membrane-bound MERTK (Sather S et al., (2007) Blood 109: 1026-1033). As a result MERTK has dual roles related to cancer progression, angiogenesis, and metastasis. On the one hand, Gas-6 activation of MERTK on endothelial cells results in inhibition of endothelial cell recruitment by cancer cells in a co-culture system. Endothelial recruitment is a key feature of cancer cells that allows for tumor angiogenesis, tumor growth, and metastasis. However, on the other hand, MERTK plays an opposite role in cancer cells, where its over-expression leads to increased metastasis, likely by releasing cleaved MERTK to generate soluble MERTK extracellular domain protein as a decoy receptor. Thus, tumor cells overexpress MERTK to promote oncogenic signaling. Also, tumor cells secrete a soluble form of the extracellular MERTK receptor that acts as a decoy receptor to reduce the ability (and/or availability) of soluble Gas-6 ligand to activate MERTK on endothelial cells, ultimately leading to endothelial recruitment, angiogenesis, and cancer progression (Png K J et al., (2012) Nature 481: 190-194).

Historically, there have been efforts to generate inhibitors of MERTK for the treatment of cancer (e.g., compound UNC1062, a potent small molecule MERTK inhibitor developed as an anticancer compound), because MERTK was thought to solely function as an oncogene (Liu J et al., (2013) Eur J Med Chem 65: 83-93; Cummings C T et al., (2013) Clin Cancer Res 19: 5275-5280; Verma A et al., (2011) Mol Cancer Ther 10: 1763-1773). In recent years, antibody-drug conjugates (ADCs) have become one of the fastest growing classes of cancer therapeutics (Beck A et al., (2017) Nat Rev Drug Discov 16: 315-337; Peters C and Brown S, (2015) Biosci Rep 35: art:e00225). ADCs comprising anti-MERTK antibodies have been described, see, e.g., International Patent Application Publications No. WO 2019/005756 and WO 2020/176497.

Anti-MERTK antibodies have been described, see, e.g., International Patent Application Publications No. WO 2016/106221, No. WO 2019/005756, and No. WO 2020/176497.

Citation of a reference herein shall not be construed as an admission that such is prior art to the present disclosure.

### 3. SUMMARY

The invention provides a polypeptide comprising a contiguous amino acid sequence of human MERTK (SEQ ID NO: 1) or a variant of said contiguous amino acid sequence, wherein the contiguous amino acid sequence comprises amino acids numbers 379-423 of the human MERTK sequence (SEQ ID NO: 1) and not more than 400 contiguous amino acids of the human MERTK sequence (SEQ ID NO: 1), and wherein the variant (a) has one or more amino acid substitutions, insertions, or deletions in the contiguous amino acid sequence relative to SEQ ID NO: 1, and (b) has at least 90% sequence identity over each of amino acid numbers 379-391 and 404-423 of SEQ ID NO: 1, or has only conservative substitutions relative to amino acid numbers 379-391 and 404-423 of SEQ ID NO: 1 and not more than two conservative substitutions relative to amino acid numbers 379-391 and 404-423 of SEQ ID NO: 1.

In a specific embodiment, the contiguous amino acid sequence comprises amino acid numbers 286-484 of SEQ ID NO: 1

In a specific embodiment, the polypeptide comprises the contiguous amino acid sequence.

In a specific embodiment, the polypeptide comprises the variant. In a specific embodiment, the variant has at least 90% sequence identity over each of amino acid numbers 379-391 and 404-423 of SEQ ID NO: 1. In a specific embodiment, the variant has only conservative substitutions relative to amino acid numbers 379-391 and 404-423 of SEQ ID NO: 1 and has not more than two conservative substitutions relative to amino acid numbers 379-391 and 404-423 of SEQ ID NO: 1. In a specific embodiment, the variant has at least 90% sequence identity over amino acid numbers 286-484 of SEQ ID NO: 1. In a specific embodiment, the variant has only conservative substitutions relative to amino acid numbers 286-484 of SEQ ID NO: 1.

In a specific embodiment, the contiguous amino acid sequence comprises not more than 300 contiguous amino acids of the human MERTK sequence (SEQ ID NO: 1).

In a specific embodiment, the contiguous amino acid sequence comprises not more than 200 contiguous amino acids of the human MERTK sequence (SEQ ID NO: 1).

In a specific embodiment, the contiguous amino acid sequence comprises not more than 100 contiguous amino acids of the human MERTK sequence (SEQ ID NO: 1).

In a specific embodiment, the contiguous amino acid sequence comprises not more than 50 contiguous amino acids of the human MERTK sequence (SEQ ID NO: 1).

In a specific embodiment, the polypeptide consists of the contiguous amino acid sequence.

In a specific embodiment, the polypeptide is a fusion protein comprising the contiguous amino acid sequence linked to a second amino acid sequence. In a specific embodiment, the second amino acid sequence comprises the amino acid sequence of an adjuvant. In a specific embodiment, the adjuvant is keyhole limpet hemocyanin. In a specific embodiment, the second amino acid sequence comprises a tag or label.

In a specific embodiment, the polypeptide is in lyophilized form.

The invention also provides a conjugate comprising the polypeptide bound to a molecule. In a specific embodiment, the molecule is an adjuvant. In a specific embodiment, the molecule is covalently bound to the polypeptide. In a specific embodiment, the conjugate is in lyophilized form.

The invention also provides an immunogenic composition comprising the polypeptide or the conjugate; and a carrier suitable for immunization purposes. In a specific embodiment, the immunogenic composition further comprises an adjuvant.

The invention also provides a method of producing an anti-MERTK antibody comprising (a) immunizing a non-human mammal with the polypeptide, the conjugate, or the immunogenic composition; (b) immortalizing antibody producing cells from the non-human mammal to produce immortalized antibody-producing cells; (c) selecting an immortalized antibody-producing cell that secretes an antibody that immunospecifically binds MERTK; and (d) culturing the immortalized antibody-producing cell in a cell culture such that antibodies are produced. In a specific embodiment, the mammal is a mouse. In a specific embodiment, the step of immortalizing antibody-producing cells is carried out by a method comprising fusing the antibody-producing cells with myeloma cells to produce antibody-producing hybridomas. In a specific embodiment, the method of producing further comprises isolating the antibodies from the cell culture. In a specific embodiment, the method of producing further comprises purifying the isolated antibodies.

The invention also provides a method of identifying antibody sequences that encode an anti-MERTK antibody or antigen-binding fragment thereof comprising (a) immunizing a non-human mammal with the polypeptide, the conjugate, or the immunogenic composition; (b) isolating antibody producing cells from the non-human mammal; (c) cloning antibody sequences of the antibody-producing cells to make a library of antibody sequences; (d) expressing antibody sequences in the library; and (e) selecting the antibody sequences that when expressed in the library produce an antibody or antigen-binding fragment thereof that immunospecifically binds to MERTK.

The invention also provides a method of screening candidate anti-MERTK antibodies or anti-MERTK antigen-binding antibody fragments comprising (a) assaying said antibodies or fragments for the ability to bind to the polypeptide or the conjugate; and (b) identifying one or more antibodies or fragments which immunospecifically bind to said polypeptide or conjugate. In a specific embodiment, the assaying of antibodies or fragments for the ability to immunospecifically bind to said polypeptide or conjugate is done using an enzyme-linked immunosorbent assay (ELISA). In a specific embodiment, the method of screening further comprises a step of assaying one or more of the antibodies or fragments which immunospecifically bind to said polypeptide or conjugate for the ability to induce internalization of MERTK on human cells; and identifying one or more antibodies or fragments that induce internalization of MERTK on human cells. In a specific embodiment, the method further comprises purifying one or more of the antibodies or fragments that induce internalization of MERTK on human cells. In a specific embodiment, the method of screening further comprises a step of assaying one or more of the antibodies or fragments that bind to said polypeptide or conjugate for the ability to induce degradation of MERTK on human cells; and identifying one or more antibodies or fragments that induce degradation of MERTK on human cells. In a specific embodiment, the method further comprises purifying one or more of the antibodies or fragments that induce degradation of MERTK on human cells. In a specific embodiment, the method of screening further comprises purifying one or more of the antibodies or fragments that immunospecifically bind to said polypeptide or conjugate.

The invention also provides a method of screening anti-MERTK antibodies or anti-MERTK antigen-binding fragments to identify an anti-MERTK antibody or anti-MERTK antigen-binding fragment that induces the internalization and/or degradation of human MERTK on human cells, the method comprising (a) assaying said antibodies or fragments for the ability to bind to the polypeptide or the conjugate; and (b) identifying one or more antibodies or fragments that immunospecifically bind to said polypeptide or conjugate, thereby identifying one or more antibodies or fragments that induce the internalization and/or degradation of human MERTK on human cells. In a specific embodiment, the method of screening further comprises assaying said one or more antibodies or fragments identified in step (b) for the ability to induce internalization and/or degradation of human MERTK on human cells; and identifying said one or more antibodies or fragments that induce internalization and/or degradation of human MERTK on human cells. In a specific embodiment, the method of screening further comprises purifying one or more of the antibodies or fragments that induce internalization of MERTK on human cells. In a specific embodiment, the method of screening further comprises purifying one or more of the antibodies or fragments that induce degradation of MERTK on human cells.

### 4. BRIEF DESCRIPTION OF FIGURES

FIG. 1 shows results of a High Mass MALDI ToF (time of flight mass spectrometry) analysis of antibody z10 at a concentration of 0.84 μM (dilution 1/8, total volume: 10 μl) before and after cross-linking with K200 (using CovalX's K200 MALDI MS analysis kit) for 180 minutes incubation time.

FIG. 2 shows results of a High Mass MALDI ToF analysis of rhMER_Fc at a concentration of 1.31 μM (dilution 4, total volume: 10 μl) before and after cross-linking with K200 for 180 minutes incubation time.

FIG. 3 shows results of a High Mass MALDI ToF analysis of rhMER_Fc (2.62 μM) cross-linked to antibody z10 (0.84 μM) (total Volume: 10 μl). Cross-linking was carried out with K200 for 360 minutes incubation time.

FIGS. 4A-4B show the overlap mapping of the trypsin, chymotrypsin, ASP-N, elastase and thermolysin peptides. Combining the peptides of trypsin, chymotrypsin, ASP-N, elastase and thermolysin proteolysis, 98.73% of the sequence is covered.

FIGS. 5A-5B show a nLC (nano liquid chromatography) chromatogram (FIG. 5A) and the total sum of the ions detected by the LTQ-Orbitrap (FIG. 5B) for trypsin digest of rhMER_Fc.

FIG. 6 shows the interaction of antibody z10 and rhMER_Fc. Amino acid numbering is according to SEQ ID NO:1.

FIG. 7 shows the interaction of antibody z10 and rhMER_Fc. FIG. 7A: ribbon/surface representation of front view; FIG. 7B: ribbon/surface representation of back view; FIG. 7C: ribbon/surface representation of side view 1; FIG. 7D: ribbon/surface representation of side view 2; FIG. 7E: ribbon/surface representation of top view; FIG. 7F: ribbon representation of front view; FIG. 7G: ribbon representation of back view; FIG. 7H: ribbon representation of side view 1; FIG. 7I: ribbon representation of side view 2; FIG. 7J: ribbon representation of top view.

### 5. DETAILED DESCRIPTION

The invention provides polypeptides that can be used as immunogens to generate anti-MERTK antibodies or antigen-binding fragments thereof, preferably anti-human MERTK antibodies and antigen-binding fragments, and in particular such antibodies that induce the internalization and/or degradation of MERTK (in particular, human MERTK) on the cell surface. Such antibodies and antigen-binding fragments thereof are contemplated for use as cancer therapeutics. The polypeptides also can be used in screening for such antibodies and antigen-binding fragments. As described herein, the polypeptides comprise a contiguous amino acid sequence of human MERTK (SEQ ID NO: 1) or a variant of said contiguous amino acid sequence. In addition to polypeptides comprising a contiguous amino acid sequence of human MERTK or variant as described herein, the invention also contemplates polypeptides consisting of, or consisting essentially of, the contiguous amino acid sequence or variant.

The sequence of human MERTK (UniProt KB Q12866) (including the signal sequence) is:

(SEQ ID NO: 1)
MGPAPLPLLLGLFLPALWRRAITEAREEAKPYPLFPGPFPGSLQTDHT
PLLSLPHASGYQPALMFSPTQPGRPHTGNVAIPQVTSVESKPLPPLAF
KHTVGHIILSEHKGVKENCSISVPNTYQDTTISWWKDGKELLGAHHAI
TQFYPDDEVTAIIASFSITSVQRSDNGSYICKMKINNEEIVSDPIYIE
VQGLPHFTKQPESMNVTRNTAFNLTCQAVGPPEPVNIFWVQNSSRVNE
QPEKSPSVLTVPGLTEMAVFSCEAHNDKGLTVSKGVQINIKAIPSPPT
EVSIRNSTAHSILISWVPGFDGYSPFRNCSIQVKEADPLSNGSVMIFN
TSALPHLYQIKQLQALANYSIGVSCMNEIGWSAVSPWILASTTEGAPS
VAPLNVTVFLNESSDNVDIRWMKPPTKQQDGELVGYRISHVWQSAGIS
KELLEEVGQNGSRARISVQVHNATCTVRIAAVTRGGVGPFSDPVKIFI
PAHGWVDYAPSSTPAPGNADPVLIIFGCFCGFILIGLILYISLAIRKR
VQETKFGNAFTEEDSELVVNYIAKKSFCRRAIELTLHSLGVSEELQNK
LEDVVIDRNLLILGKILGEGEFGSVMEGNLKQEDGTSLKVAVKTMKLD
NSSQREIEEFLSEAACMKDFSHPNVIRLLGVCIEMSSQGIPKPMVILP
FMKYGDLHTYLLYSRLETGPKHIPLQTLLKFMVDIALGMEYLSNRNFL
HRDLAARNCMLRDDMTVCVADFGLSKKIYSGDYYRQGRIAKMPVKWIA
IESLADRVYTSKSDVWAFGVTMWEIATRGMTPYPGVQNHEMYDYLLHG
HRLKQPEDCLDELYEIMYSCWRTDPLDRPTFSVLRLQLEKLLESLPDV
RNQADVIYVNTQLLESSEGLAQGSTLAPLDLNIDPDSIIASCTPRAAI
SVVTAEVHDSKPHEGRYILNGGSEEWEDLTSAPSAAVTAEKNSVLPGE
RLVRNGVSWSHSSMLPLGSSLPDELLFADDSSEGSEVLM

### 5.1 Polypeptides

As used herein, the term “polypeptide” includes proteins as well as peptides.

As used herein, when referring to “human MERTK” or “MERTK,” unless the context indicates otherwise, such is deemed to be the mature form of human MERTK or MERTK, respectively, which lacks the signal sequence. The signal sequence of human MERTK consists of amino acids 1-20 of SEQ ID NO: 1. Thus, the amino acid sequence of the mature form of human MERTK is amino acid numbers 21-999 of SEQ ID NO: 1. In a specific embodiment, MERTK, as referred to herein, is human MERTK (unless the context indicates otherwise).

The invention provides a polypeptide comprising a contiguous amino acid sequence of human MERTK (SEQ ID NO: 1) or a variant of said contiguous amino acid sequence, wherein the contiguous amino acid sequence comprises amino acids numbers 379-423 of the human MERTK sequence (SEQ ID NO: 1) and not more than 400 contiguous amino acids of the human MERTK sequence (SEQ ID NO: 1), and wherein the variant (a) has one or more amino acid substitutions, insertions, or deletions in the contiguous amino acid sequence relative to SEQ ID NO: 1, and (b) has at least 70% (e.g. at least 90%) sequence identity over each of amino acid numbers 379-391 and 404-423 of SEQ ID NO: 1, or has only conservative substitutions relative to amino acid numbers 379-391 and 404-423 of SEQ ID NO: 1 and not more than ten (e.g., not more than two) conservative substitutions relative to amino acid numbers 379-391 and 404-423 of SEQ ID NO: 1.

In a specific embodiment, the polypeptide comprises the contiguous amino acid sequence (and not the variant). In another specific embodiment, the polypeptide comprises the variant.

The polypeptide of the invention comprises less than the full-length extracellular domain sequence of human MERTK, and lacks the signal sequence of human MERTK.

In a specific embodiment, the polypeptide comprises the contiguous amino acid sequence (and not the variant).

In a specific embodiment, the contiguous amino acid sequence comprises not more than 300 contiguous amino acids of the human MERTK sequence (SEQ ID NO: 1). In a specific embodiment, the contiguous amino acid sequence comprises not more than 200 contiguous amino acids of the human MERTK sequence (SEQ ID NO: 1). In a specific embodiment, the contiguous amino acid sequence comprises not more than 100 contiguous amino acids of the human MERTK sequence (SEQ ID NO: 1). In a specific embodiment, the contiguous amino acid sequence comprises not more than 90 contiguous amino acids of the human MERTK sequence (SEQ ID NO: 1). In a specific embodiment, the contiguous amino acid sequence comprises not more than 80 contiguous amino acids of the human MERTK sequence (SEQ ID NO: 1). In a specific embodiment, the contiguous amino acid sequence comprises not more than 70 contiguous amino acids of the human MERTK sequence (SEQ ID NO: 1). In a specific embodiment, the contiguous amino acid sequence comprises not more than 60 contiguous amino acids of the human MERTK sequence (SEQ ID NO: 1). In a specific embodiment, the contiguous amino acid sequence comprises not more than 50 contiguous amino acids of the human MERTK sequence (SEQ ID NO: 1)

In a specific embodiment, the contiguous amino acid sequence consists of 50, 60, 70, 80, 90 100, 200, 300, or 400 contiguous amino acids of the human MERTK sequence (SEQ ID NO: 1). In a specific embodiment, the contiguous amino acid sequence consists of amino acid numbers 379-423 of SEQ ID NO: 1.

In a specific embodiment, a polypeptide provided herein is capable of forming a three-dimensional structure that is the same or similar to a three-dimensional structure contained within native full-length human MERTK (e.g., that formed by the fibronectin type-III domains of MERTK). The two fibronectin type-III domains of human MERTK correspond to amino acid numbers 286-381 and 386-484 of SEQ ID NO: 1, respectively. Any suitable technique known to one of skill in the art can be used to evaluate structural similarity. For example, binding, e.g. under non-denaturing conditions, to an antibody that recognizes native MERTK, or molecular modeling, might be used to indicate structural similarity.

In a specific embodiment, the contiguous amino acid sequence comprises the two fibronectin domains of human MERTK, which are amino acids 286-381 and amino acids 386-484 of SEQ ID NO: 1, respectively. In a specific embodiment, the contiguous amino acid sequence comprises the two fibronectin domains of human MERTK and the intervening amino acids (i.e., the contiguous amino acid sequence comprises amino acids 286-484 of SEQ ID NO: 1).

In a specific embodiment, the polypeptide has at least 80% sequence identity over amino acid numbers 286-484 of SEQ ID NO: 1. In a specific embodiment, the polypeptide has at least 90% sequence identity over amino acid numbers 286-484 of SEQ ID NO: 1. In a specific embodiment, the polypeptide has at least 95% sequence identity over amino acid numbers 286-484 of SEQ ID NO: 1. In a specific embodiment, the polypeptide has at least 99% sequence identity over amino acid numbers 286-484 of SEQ ID NO: 1.

In a specific embodiment, the polypeptide comprises the variant of the contiguous amino acid sequence of human MERTK.

In a specific embodiment, the variant has one or more amino acid substitutions, insertions, or deletions in the contiguous amino acid sequence relative to SEQ ID NO: 1 and has at least 70% sequence identity over each of amino acid numbers 379-391 and 404-423 of SEQ ID NO: 1. In a specific embodiment, the variant has one or more amino acid substitutions, insertions, or deletions in the contiguous amino acid sequence relative to SEQ ID NO: 1 and has least 80% sequence identity over each of amino acid numbers 379-391 and 404-423 of SEQ ID NO: 1. In a specific embodiment, the variant has one or more amino acid substitutions, insertions, or deletions in the contiguous amino acid sequence relative to SEQ ID NO: 1 and has at least 90/o sequence identity over each of amino acid numbers 379-391 and 404-423 of SEQ ID NO: 1. In a specific embodiment, the variant has one or more amino acid substitutions, insertions, or deletions in the contiguous amino acid sequence relative to SEQ ID NO: 1 and has at least 95% sequence identity over each of amino acid numbers 379-391 and 404-423 of SEQ ID NO: 1. In a specific embodiment, the variant has one or more amino acid substitutions, insertions, or deletions in the contiguous amino acid sequence relative to SEQ ID NO: 1 and has at least 99% sequence identity over each of amino acid numbers 379-391 and 404423 of SEQ ID NO: 1.

In a specific embodiment, the variant has at least 80% sequence identity over amino acid numbers 286-484 of SEQ ID NO: 1. In a specific embodiment, the variant has at least 90% sequence identity over amino acid numbers 286484 of SEQ ID NO: 1. In a specific embodiment, the variant has at least 95% sequence identity over amino acid numbers 286-484 of SEQ ID NO. 1 In a specific embodiment, the variant has at least 99% sequence identity over amino acid numbers 286-484 of SEQ ID NO: 1.

The determination of percent identity between two sequences (e.g., amino acid sequences) can be accomplished using any algorithm known in the art. A specific, non-limiting example of an algorithm utilized for the comparison of two sequences is the algorithm of Karlin S & Altschul S F (1990) PNAS 87: 2264-2268, modified as in Karlin S & Altschul S F (1993) PNAS 90: 5873-5877. Such an algorithm is incorporated into the NBLAST and XBLAST programs of Altschul S F et al., (1990) J Mol Biol 215: 403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than ten (e.g., one, two, three, four, five, six, seven, eight, nine, or ten) conservative substitutions in amino acid numbers 379-391 and 404-423 of SEQ ID NO: 1. In a specific embodiment, the contiguous amino acid sequence comprises amino acid numbers 286-484 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than nine (e.g., one, two, three, four, five, six, seven, eight, or nine) conservative substitutions in amino acid numbers 379-391 and 404-423 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than eight (e.g., one, two, three, four, five, six, seven, or eight) conservative substitutions in amino acid numbers 379-391 and 404-423 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than seven (e.g., one, two, three, four, five, six, or seven) conservative substitutions in amino acid numbers 379-391 and 404-423 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than six (e.g., one, two, three, four, five, or six) conservative substitutions in amino acid numbers 379-391 and 404-423 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than five (e.g., one, two, three, four, or five) conservative substitutions in amino acid numbers 379-391 and 404-423 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than four (e.g., one, two, three, or four) conservative substitutions in amino acid numbers 379-391 and 404-423 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than three (e.g., one, two, or three) conservative substitutions in amino acid numbers 379-391 and 404-423 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than two (e.g., one or two) conservative substitutions in amino acid numbers 379-391 and 404-423 of SEQ ID NO: 1.

In a specific embodiment, the variant has one or more amino acid substitutions, insertions, or deletions in the contiguous amino acid sequence relative to SEQ ID NO: 1, and has only conservative substitutions relative to amino acid numbers 379-391 and 404-423 of SEQ ID NO: 1 and has not more than one conservative substitution in amino acid numbers 379-391 and 404-423 of SEQ ID NO: 1.

The term “conservative substitution” (i.e., “conservative amino acid substitution”) can have any meaning known in the art. A conservative amino acid substitution is a substitution of one amino acid with another amino acid which has similar physico-chemical properties (e.g., a similar charge and size). Groups of amino acids that have similar charges are well known in the art and include, for example, the following six conservative substitution groups: Group 1 (alanine, glycine, serine, and threonine), Group 2 (aspartic acid and glutamic acid), Group 3 (asparagine and glutamine), Group 4 (arginine, lysine, and histidine), Group 5 (isoleucine, leucine, methionine, and valine), and Group 6 (phenylalanine, tyrosine and tryptophan).

Amino acids may also be grouped into conservative substitution groups by similar function, chemical structure, or composition (e.g., acidic, basic, aliphatic, aromatic, sulfur-containing), such as the following groups. Aliphatic amino acids include, for example, glycine, alanine, valine, leucine, and isoleucine. Sulfur-containing amino acids include, for example, methionine and cysteine. Acidic amino acids and their amides include, for example, aspartic acid, glutamic acid, asparagine, and glutamine. Amino acids with small aliphatic, nonpolar or slightly polar residues include, for example, alanine, serine, threonine, proline, and glycine. Amino acids with polar, negatively charged residues and their amides include, for example, aspartic acid, asparagine, glutamic acid, and glutamine. Amino acids with polar, positively charged residues include for example, histidine, arginine, and lysine. Amino acids with large aliphatic, nonpolar residues include, for example, methionine, leucine, isoleucine, valine, and cysteine. Amino acids with large aromatic residues include, for example, phenylalanine, tyrosine, and tryptophan.

Generally, a conservative amino acid substitution is not expected to influence the stability or function of the protein.

A polypeptide provided herein may be modified, e.g., at the N-terminus, C-terminus or internally, by one or more modifications to polypeptides known in the art.

In a specific embodiment, a polypeptide provided herein contains one or more N-terminal modifications. In a specific embodiment, a polypeptide provided herein contains one or more C-terminal modifications. In a specific embodiment, a polypeptide provided herein contains one or more internal modifications.

For example, in certain embodiments, a polypeptide provided herein is modified by disulfide bond formation, glycosylation (e.g., N-linked glycosylation), farnesylation, lipid modification (e.g. S-palmitoylation), acetylation, biotinylation, phosphorylation, fusion at the N- or C-terminus to a sequence of a different polypeptide, or conjugation to a different molecule.

In certain embodiments, modification of the N-terminus includes acylation including N-formyl, N-acetyl, N-propyl, and long chain fatty acid groups.

In a specific embodiment, the C-terminus is a carboxylic acid. In a specific embodiment, modification of the C-terminus is by amidation. Thus, in a specific embodiment, the C-terminus is an amide.

In a specific embodiment, one or more L-amino acids in a polypeptide described herein are substituted with D-amino acid(s). The D-amino acid can be the same amino acid as the amino acid residue being substituted, or can be a different amino acid.

In a specific embodiment, one or more amino acids of a polypeptide provided herein are substituted with a modified amino acid that is a non-standard amino acid.

In another specific embodiment, a polypeptide provided herein is cyclized, using any suitable method known in the art, including but not limited to peptide or non-peptide linkers (for example, alanine bridges) to achieve cyclization. For example, a polypeptide provided herein may be cyclized to mimic a three dimensional structure of the native human MERTK peptide.

In a specific embodiment, the polypeptide is a fusion protein comprising a contiguous amino acid sequence of the human MERTK sequence (e.g., 50, 100, 200, 300 or 400 contiguous amino acids of SEQ ID NO: 1) linked to a second amino acid sequence.

In a specific embodiment, the second amino acid sequence is the amino acid sequence of an adjuvant. In a specific embodiment, the adjuvant is keyhole limpet hemocyanin.

In a specific embodiment, the second amino acid sequences comprises a tag or label. Examples of tags or labels include His-tags, Fc-tags, GST tags, FLAG tags, and Myc tags.

In another aspect, provided herein is a conjugate comprising a polypeptide described herein bound to a molecule. The molecule differs from the polypeptide. The molecule can be covalently or noncovalently bound to the polypeptide. In a specific embodiment, the molecule is covalently bound to the polypeptide. The molecule can be bound to the polypeptide at the N-terminus, or C-terminus, or at an internal position in the polypeptide. In a specific embodiment, for example, when the conjugate is used in immunization, the molecule is an adjuvant (e.g., keyhole limpet hemocyanin). In a specific embodiment, a conjugate provided herein may be in lyophilized form. A lyophilized conjugate may be reconstituted in a carrier suitable for immunization purposes, e.g., a sterile solution, to form an immunogenic composition, before being used for immunization. In another specific embodiment, for example, when the conjugate is used in screening antibodies or antigen-binding fragment, the molecule is a label, which can be a peptide or non-peptide label. The label can be, but is not limited to a fluorescent moiety, biotin, an enzymatic moiety, etc.

The polypeptides and conjugates provided herein may be made using any suitable method known in the art. In a specific embodiment, a polypeptide described herein is synthesized by chemical synthetic methods. In a specific embodiment, a polypeptide provided herein is recombinantly expressed using a bacterial, yeast, plant, mammalian, or other expression system in vitro.

In a specific embodiment, a polypeptide described herein is synthesized using solid-phase synthesis or other chemical syntheses. The polypeptide can be prepared via a solid-phase synthesis procedure such as described in Barany, G. and Merrifield, R. B. The Peptides, Gross E., Meienhofer, J. Eds., Academic Press: New York, 1980, vol. 2, pp. 1-284; Solid phase synthesis: A practical guide, S. A. Kates, F. Albericio, Eds. Marcel Dekker: New York, 2000; Myers A. G. et al. (1997) J. Amer. Chem. Soc. 119:656; Myers A. G. et al. (1999) J. Org. Chem. 64:3322D; A. Wellings, E. Atherton, (1997) Methods Enzymol. 289:44; Fields, G. B. et al., (1990) Int. J. Peptide Protein Res. 35:161; H. Rink, (1987) Tetrahedron Lett. 28: 3787; R. C. Sheppard, B. J. Williams, (1982) Int. J. Rept. Protein Res. 20:451; J. Coste, et al., (1991) Tetrahedron Lett. 32:1967; L. A. Carpino, A. Elfaham, C. A. Minor, F. Albericio, (1994) J. Chem, Soc. Chem. Comm., 201; M. Felix, et al., (1998) J. Peptide Res. 52:155; U.S. Pat. No. 5,770,732 issued Jun. 23, 1998; U.S. Pat. No. 5,514,814 issued May 7, 1996; and U.S. Pat. No. 5,489,692 issued Feb. 6, 1996. Starting materials useful for preparing the polypeptides of the invention, and intermediates therefore, are commercially available or can be prepared from commercially available materials using known synthetic methods and reagents.

The synthesized polypeptide can be characterized by any suitable analytic method such as analytical HPLC, FAB-MS, ES-MS and/or amino acid analysis. In a specific embodiment, the polypeptide has greater than 97% purity, e.g., as determined from all UV active peaks.

If desired, non-natural, non-alpha amino acids and peptide mimetics may be incorporated into the polypeptide during synthesis.

In a specific embodiment, a polypeptide provided herein may be in lyophilized form. A lyophilized polypeptide may be reconstituted in a carrier suitable for immunization purposes, e.g., a sterile solution, to form an immunogenic composition before being used for immunization.

### 5.2 Methods of Producing Antibodies

In another aspect, provided herein are methods of producing an anti-MERTK antibody or an anti-MERTK antigen-binding antibody fragment. An anti-MERTK antibody or an antigen-binding fragment produced in accordance with a method described herein is indicated for use in the treatment of cancer.

As used herein, the term “antigen-binding fragment” or “antigen-binding antibody fragment” refers to a portion of an antibody molecule which comprises the amino acid residues that confer on the antibody molecule its specificity for the antigen (e.g., the complementarity determining regions (CDRs) surrounded by framework regions). By way of example, antigen-binding fragments include Fab fragments, F(ab′)₂ fragments, nanobodies, antigen-binding peptides, and single-chain Fvs (scFvs). A scFv is a protein comprising an antibody variable heavy chain region and antibody variable light chain region connected by a peptide linker.

Antigen-binding fragments can be generated by any technique known to those of skill in the art. For example, Fab and F(ab′)₂ fragments can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region. A scFv can be generated by recombinant methods known in the art. A nanobody can be made by methods known in the art; see e.g., Yang E. Y. & Shah K., Front. Oncol., 2020, 10:1182 (https://doi.org/10.3389/fonc.2020.01182); Muyldermans S., The FEBS Journal, 2021, 288:2084-2102; and Harmsen M. M. & De Haard H. J., Appl Microbiol Biotechnol, 2007, 77:13-22. An antigen-binding peptide can be made by methods known in the art, see e.g., Saw P. E. & Song E W., Protein Cell, 2019, 10:787-807.

In another aspect, provided herein is an immunogenic composition comprising a polypeptide or a conjugate provided herein and a carrier suitable for immunization purposes. In a specific embodiment, the immunogenic composition further comprises an adjuvant. Such immunogenic compositions may be used in immunization to produce anti-MERTK antibodies. Specific examples of adjuvants that may be used include, but are not limited to, aluminum salts (e.g., such as aluminum hydroxide and aluminum phosphate), emulsion-based adjuvants (e.g., Freund's Complete Adjuvant, MF59), TLR agonists (e.g., monophosphoryl lipid A, polyI:C, remiquimod, and imiquimod). Other examples of adjuvants are described, e.g., in McKee and Marrack, Curr Opin Immunol. 2017 August; 47: 44-51. The immunogenic composition may be used to immunize a non-human mammal (e.g., a mouse, a rat, or a rabbit). In a specific embodiment, a method of producing an anti-MERTK antibody provided herein comprises more than one immunization of a non-human mammal. For example, a polypeptide provided herein may be administered to the non-human mammal repeatedly over the course of several days (e.g., about 7 days, about 10 days, about 14 days, or about 21 days). In a specific embodiment, the non-human mammal is immunized twice, three times, four times, or five times.

The carrier suitable for immunization purposes, of the immunogenic composition, can be any suitable carrier known in the art. In a specific embodiment, an immunogenic composition is a solution. In a specific embodiment, the carrier is a sterile carrier, e.g., the immunogenic composition is a sterile solution in which the polypeptide or conjugate is dissolved. In a specific embodiment, the immunogenic composition comprises the polypeptide or conjugate dissolved in a carrier that is a suitable buffer. Examples of buffers which may be used for an immunogenic composition include, without limitation, acetate, citrate, histidine, succinate, phosphate, hydroxymethylaminomethane and (Tris) buffers, preferably containing a physiologic level of saline (150 mM NaCl). In a specific embodiment, the immunogenic composition comprises a polypeptide or conjugate in a solution of 20 mM Histidine, 150 mM NaCl, pH 6.2. In a specific embodiment, a polypeptide or conjugate provided herein is in lyophilized form, and is reconstituted in a carrier suitable for immunization purposes, e.g., a sterile solution, to form an immunogenic composition before being used for immunization.

An anti-MERTK antibody produced in accordance with the methods described herein may be, for example, a monoclonal antibody. Monoclonal antibodies can be prepared using a wide variety of techniques known in the art including the use of hybridoma, recombinant, and phage display technologies, or a combination thereof. Methods for the preparation of clonal cell lines and of monoclonal antibodies expressed thereby are well known in the art (see, for example, Chapter 11 in Short Protocols in Molecular Biology, (2002) 5th Ed., Ausubel F M et al., supra). For example, monoclonal antibodies can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981); and Kohler G & Milstein C (1975) Nature 256: 495.

In a specific embodiment, provided herein is a method of producing an anti-MERTK antibody comprising (a) immunizing a non-human mammal with a polypeptide, a conjugate, or an immunogenic composition provided herein; (b) immortalizing antibody-producing cells from the non-human mammal to produce immortalized antibody-producing cells; (c) selecting an immortalized antibody-producing cell that secretes an antibody that immunospecifically binds MERTK and/or said polypeptide or conjugate; (d) culturing the immortalized antibody-producing cell in a cell culture such that antibodies are produced. In a specific embodiment, the immunizing is by injecting into the animal. In a specific embodiment, the mammal is a rodent (e.g., a rat, a rabbit, or a mouse). In a specific embodiment, the mammal is a camelid (e.g., a camel or a llama). In a specific embodiment, the immortalized antibody-producing cell is a hybridoma. In a specific embodiment, the step of immortalizing antibody-producing cells is carried out by a method comprising fusing the antibody-producing cells with myeloma cells to produce antibody-producing hybridomas. Thus, in a specific embodiment, a method of producing an anti-MERTK antibody described herein comprises producing an antibody using hybridoma technology.

Methods for producing and screening for specific antibodies using hybridoma technology are routine and well known in the art. A specific embodiment is described as follows. A mouse (or other non-human mammal, such as, for example, a rat, monkey, donkey, pig, sheep, hamster, or dog) can be immunized with an antigen (a polypeptide or conjugate described herein) and once an immune response is detected, e.g., antibodies specific for the antigen are detected in the serum, the spleen is harvested and splenocytes (containing antibody-producing cells) isolated. The splenocytes are then fused by well-known techniques to any suitable myeloma cells, for example cells from cell line SP2/0 available from the American Type Culture Collection (ATCC®) (Manassas, VA), to form hybridomas. Hybridomas are selected and cloned by limited dilution. The hybridoma cells thus prepared are seeded and grown in a suitable culture medium that preferably contains one or more substances that inhibit the growth or survival of the unfused, parental myeloma cells. Culture medium in which hybridoma cells are growing is assayed for production of monoclonal antibodies directed against MERTK. After hybridoma cells that produce antibodies of the desired specificity, affinity, and/or activity are identified, the clones may be subcloned, grown, and separated from the culture medium by standard methods (Goding J W (Ed), Monoclonal Antibodies: Principles and Practice, supra). The binding specificity of monoclonal antibodies produced by hybridoma cells is determined by a method known in the art, for example, immunoprecipitation or by an in vitro binding assay, such as, for example, radioimmunoassay (RIA) or enzyme-linked immunoabsorbent assay (ELISA).

In a specific embodiment, the antibody that is produced is an immunoglobulin. The antibody produced in accordance with the methods described herein can be from any class of immunoglobulins, including IgM, IgG, IgD, IgA and IgE, and any isotype, including IgG₁, IgG₂, IgG₃ and IgG₄. In a specific embodiment, screening for a particular isotype may be carried out by assaying produced antibodies for binding to an antibody or antiserum that recognizes the constant domain of the particular isotype. In a specific embodiment, a produced or identified anti-MERTK antibody can recombinantly attached to a desired constant region, e.g. of a particular isotype.

In a specific embodiment, provided herein is a method of identifying antibody sequences that encode an anti-MERTK antibody or antigen-binding fragment thereof comprising (a) immunizing a non-human mammal with a polypeptide, a conjugate, or an immunogenic composition provided herein; (b) isolating antibody producing cells from the non-human mammal; (c) cloning antibody sequences of the antibody-producing cells to make a library of antibody sequences; (d) expressing antibody sequences in the library; and (e) selecting the antibody sequences that when expressed in the library produce an antibody or antigen-binding fragment thereof that immunospecifically binds to MERTK and/or said polypeptide or conjugate. In a specific embodiment, the mammal is a rodent (e.g., a rat, a rabbit, or a mouse). In a specific embodiment, the mammal is a camelid (e.g., a camel or a llama). The antibody sequences that are cloned and expressed in the library may be VH and/or VL sequences. In a specific embodiment, the library is a scFv library.

The sequences of the antibody-producing cells may be determined by any suitable method known in the art, including, for example, DNA sequencing. In a specific embodiment, the library of antibody sequences is a phage display library. In a specific embodiment, the library is a scFv library. In a specific embodiment, the scFv library is a yeast scFv library.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated into E. coli cells and the E. coli is infected with helper phage. Phage used in these methods are typically filamentous phage including fd and M13, and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make antibodies include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, or any desired antigen-binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax R L et al., (1992) BioTechniques 12(6): 864-9; Sawai H et al., (1995) Am J Reprod Immunol 34: 26-34; and Better M et al., (1988) Science 240: 1041-1043.

In a specific embodiment, an anti-MERTK antibody or antigen-binding fragment produced in accordance with the methods described herein is a human antibody or antigen-binding fragment thereof. Human antibodies can be produced using any method known in the art.

For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., a polypeptide described herein. Monoclonal antibodies directed against the antigen can be obtained from the immunized, transgenic mice using single B cell or hybridoma technology. The human immunoglobulin transgenes harbored by the transgenic mice rearrange during B cell differentiation, and subsequently undergo class switching recombination and somatic hyper-mutation. Thus, using such a technique, it is possible to produce therapeutically useful IgG, IgA, IgM and IgE antibodies. For an overview of this technology for producing human antibodies, see, e.g., Lonberg N & Huszar D (1995) Int Rev Immunol 13:65-93. For a detailed discussion of this technology for producing human antibodies and human monoclonal antibodies and protocols for producing such antibodies, see, e.g., International Publication Nos. WO 98/24893, WO 96/34096 and WO 96/33735; and U.S. Pat. Nos. 5,413,923, 5,625,126, 5,633,425, 5,569,825, 5,661,016, 5,545,806, 5,814,318 and 5,939,598. Examples of mice capable of producing human antibodies include the Trianni® mouse (described in, e.g., U.S. Pat. Nos. 10,881,084 and 10,793,829), the Xenomouse™ (Abgenix, Inc.; U.S. Pat. Nos. 6,075,181 and 6,150,184), the HuAb-Mouse™ (Medarex, Inc./Gen Pharm; U.S. Pat. Nos. 5,545,806 and 5,569,825), the Trans Chromo Mouse™ (Kirin) and the KM Mouser™ (Medarex/Kirin).

In a specific embodiment, an anti-MERTK antibody or an antigen-binding fragment thereof produced in accordance with the methods described herein is further modified, e.g., to produce a chimeric antibody or a humanized antibody. A chimeric antibody is a molecule in which different portions of the antibody are derived from different immunoglobulin molecule while a humanized antibody comprises a framework region having substantially the amino acid sequence of a human immunoglobulin.

In a specific embodiment, an anti-MERTK antibody or an antigen-binding fragment there of produced in accordance with the methods described herein is further modified, e.g., to produce a multispecific (e.g., bispecific) antibody.

In a specific embodiment, the methods of producing an anti-MERTK antibody provided herein further comprise isolating the antibody(ies) from the cell culture. Once an anti-MERTK antibody or an antigen-binding fragment thereof has been produced and isolated from the cell culture, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification. In specific embodiments, an anti-MERTK antibody or an antigen-binding fragment thereof is isolated or purified. In a specific embodiment, an anti-MERTK antibody or an antigen-binding fragment thereof is substantially free of other antibodies with different antigenic specificities than the isolated antibody. For example, in a particular embodiment, a preparation of an antibody described herein is substantially free of cellular material and/or chemical precursors. In a specific embodiment, an antibody preparation that is “substantially free of cellular material” includes preparations of an antibody in which the antibody is separated from cellular components of the cells from which it is isolated. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, antibody fragments.

### 5.3 Methods of Screening Antibodies

In another aspect, provided herein is a method of screening candidate anti-MERTK antibodies or anti-MERTK antigen-binding fragments, the method comprising (a) assaying said antibodies or fragments for the ability to bind to a polypeptide or a conjugate provided herein; and (b) identifying one or more antibodies or antigen-binding fragments which immunospecifically bind to said polypeptide or conjugate. In a specific embodiment, the candidate anti-MERTK antibodies or antigen-binding fragments are produced or identified as described herein above. In a specific embodiment, the candidate anti-MERTK antibodies or antigen-binding fragments are a library of scFvs, nanobodies, or peptide antibodies. In a specific embodiment, the candidate antibodies or antigen-binding fragments are a phage display or ribosome display or yeast display library, for example, of human antibody sequences. The assaying of antibodies or antigen-binding fragments for the ability to immunospecifically bind to said polypeptide or conjugate can be done by any suitable method known in the art. In a specific embodiment, the assaying of antibodies or antigen-binding fragments for the ability to immunospecifically bind to said polypeptide or conjugate is done using an enzyme-linked immunosorbent assay (ELISA).

In a specific embodiment, a polypeptide used in a method of screening provided herein is a fusion protein as described herein, wherein the second amino acid sequence comprises a tag or label. In another specific embodiment, a conjugate provided herein is used in a method of screening provided herein, and comprises a polypeptide as described herein bound to a molecule that is a tag or label.

In a specific embodiment, a method of screening provided herein further comprises a step of assaying one or more of the antibodies or fragments which immunospecifically bind to said polypeptide or conjugate for the ability to induce internalization of MERTK on human cells; and identifying one or more antibodies or fragments that induce internalization of MERTK on human cells.

In a specific embodiment, a method of screening provided herein further comprises a step of assaying one or more of the antibodies or fragments that bind to said polypeptide or conjugate for the ability to induce degradation of MERTK on human cells; and identifying one or more antibodies or fragments that induce degradation of MERTK on human cells.

In another aspect, provided herein is a method of screening anti-MERTK antibodies or anti-MERTK antigen-binding fragments to identify an anti-MERTK antibody or anti-MERTK antigen-binding fragment that induces the internalization and/or degradation of human MERTK on human cells, the method comprising (a) assaying said antibodies or fragments for the ability to bind to a polypeptide or a conjugate provided herein; and (b) identifying one or more antibodies or fragments that immunospecifically bind to said polypeptide or conjugate, thereby identifying one or more antibodies or fragments that induce the internalization and/or degradation of human MERTK on human cells. In a specific embodiment, the method of screening further comprises assaying said one or more antibodies or fragments identified in step (b) for the ability to induce internalization and/or degradation of human MERTK on human cells; and identifying said one or more antibodies or fragments that induce internalization and/or degradation of human MERTK on human cells.

In a specific embodiment, a method of screening provided herein further comprises purifying one or more of the antibodies or fragments that immunospecifically bind to said polypeptide or conjugate.

In a specific embodiment, a method of screening provided herein further comprises purifying one or more of the antibodies or fragments that induce internalization of MERTK in human cells.

In a specific embodiment, a method of screening provided herein further comprises purifying one or more of the antibodies or fragments that induce degradation of MERTK in human cells.

Methods of determining whether an antibody induces the internalization and/or degradation of human MERTK on human cells are known in the art and have been described, for example, in paragraphs 430 and 442 of International Patent Application Publication No. WO 2020/176497. For example, the antibodies being screened may be labeled with pHrodo Red, a pH-sensitive dye, and internalization of the antibody may be determined by flow cytometry detecting pHrodo fluorescence, which is minimal at neutral pH and maximal in acidic environments, such as the lysosomes. Degradation of MERTK may be determined by measuring changes in MERTK protein expression, e.g., using Western Blotting.

### 6. EXAMPLES

The following example is offered by way of illustration and not by way of limitation.

### 6.1 Example 1: Determination of Antibody Z10 Epitope

The epitope to which antibody z10 binds was determined using High-Mass MALDI mass spectrometry. Antibody z10 is a humanized anti-human MERTK monoclonal antibody described in, e.g., International Patent Application Publication No. WO 2020/176497, which is incorporated by reference herein in its entirety. Antibody z10 has been shown to induce internalization of human MERTK on human cells, and to induce degradation of human MERTK on human cells (see WO 2020/176497, paragraphs 441-443 and 483). Antibody z10 also has been shown to inhibit colony formation and cell survival of cancer cells in cell culture (see WO 2020/176497, paragraphs 445 and 452), and to result in tumor reduction in a mouse breast cancer model in vivo (see WO 2020/176497, paragraph 453).

As described in this Example, the epitope to which antibody z10 binds was determined using High-Mass MALDI mass spectrometry.

### 6.1.1 High-Mass MALDI Mass Spectrometry

A high-mass MALDI analysis was performed on each sample antibody z10 (20 mM histidine+150 mM NaCl; 7.37 mg/mL; 50 μl; pH 6.2) and recombinant human MERTK fused to the Fc domain (rhMER_Fc) (reconstituted at 100 μg/ml in sterile PBS; 2×lyophilized) in order to verify their integrity and aggregation level. The rhMER-Fc was obtained from R&D Systems (catalog number 891-MR) and consists of amino acid numbers Arg26 to Ala499 of human MERTK (SEQ ID NO:1) fused via a Ile-Glu-Gly-Arg-Met-Asp amino acid linker fused to amino acids Pro100 to Lys330 of human IgG1.

### 6.1.1.1 Materials and Methods

### (i) Instrumentation

For the integrity/aggregation test, the measurements were performed using an Autoflex II MALDI ToF mass spectrometer (Bruker) equipped with CovalX's HM4 interaction module. CovalX's interaction module contains a special detecting system designed to optimize detection up to 2 MDa with nano-molar sensitivity.

### (ii) Sample Preparation:

### (a) Control Experiments

rhMER_Fc protein sample was dissolved with distilled water to reach a concentration of 1 mg/ml, as shown in Table 1:

TABLE 1

| | |
|---|---|
| Sample initial | Sample final |

| | Powder | Volume | Conc. |
|---|---|---|---|
| rhMER_Fc | 100 μg | 100 μl | 1 mg/ml |

20 μl of each protein sample antibody z10 and rhMER_Fc were pipetted to prepare 8 dilutions with final volume 10 μl. These 8 dilutions of the samples were prepared in order to obtain the expected concentrations shown in Table 2:

TABLE 2

| | Antibody z10 | | | rhMER_Fc | | |
|---|---|---|---|---|---|---|
| Dilution | Volume | Conc. | | Volume | Conc. | |
| 1 | 10 μl | 1 | mg/ml | 10 μl | 1 | mg/ml |
| 1/2 | 10 μl | 0.5 | mg/ml | 10 μl | 0.5 | mg/ml |
| 1/4 | 10 μl | 250 | μg/ml | 10 μl | 250 | μg/ml |
| 1/8 | 10 μl | 125 | μg/ml | 10 μl | 125 | μg/ml |
| 1/16 | 10 μl | 62.5 | μg/ml | 10 μl | 62.5 | μg/ml |
| 1/32 | 10 μl | 31.25 | μg/ml | 10 μl | 31.25 | μg/ml |
| 1/64 | 10 μl | 15.6 | μg/ml | 10 μl | 15.6 | μg/ml |
| 1/128 | 10 μl | 7.8 | μg/ml | 10 μl | 7.8 | μg/ml |

1 μl of each dilution obtained was mixed with 1 μl of a matrix composed of a re-crystallized sinapinic acid matrix (10 mg/ml) in acetonitrile/water (1:1, v/v), TFA 0.1% (K200 MALDI Kit). After mixing, 1 μl of each sample was spotted on the MALDI plate (SCOUT 384). After crystallization at room temperature, the plate was introduced in the MALDI mass spectrometer and analyzed immediately in High-Mass MALDI mode. The analysis has been repeated in triplicate.

### (b) Cross-Link Experiments

The cross-linking experiments allow the direct analysis of non-covalent interaction by High-Mass MALDI mass spectrometry. By mixing a protein sample containing non covalent interactions with a specially developed cross-linking mixture (Bich, C et al. Anal. Chem., 2010, 82 (1), pp 172-179), it is possible to specifically detect non covalent complex with high-sensitivity. The covalent binding generated allows the interacting species to survive the sample preparation process and the MALDI ionization. A special High-Mass detection system allows characterizing the interaction in the High-Mass range.

Each mixture prepared for the control experiment (9 μl left) was submitted to cross-linking using CovalX's K200 MALDI MS analysis kit. 9 μl of the mixtures (from 1 to 1/128) were mixed with 1 μl of K200 Stabilizer reagent (2 mg/ml) and incubated at room temperature. After the incubation time (180 minutes) the samples were prepared for MALDI analysis as for Control experiments. The samples were analyzed by High-Mass MALDI analysis immediately after crystallization.

(iii) High-Mass MALDI MS Analysis

The MALDI ToF MS analysis was performed using CovalX's HM4 interaction module with a standard nitrogen laser and focusing on different mass ranges from 0 to 1500 kDa.

For the analysis, the following parameters were applied:
Mass Spectrometer:
Linear and Positive mode
   Ion Source 1: 20 kV
   Ion Source 2: 17 kV
   Lens: 12 kV
   Pulse Ion Extraction: 400 ns
HM4:
   Gain Voltage: 3.14 kV
   Acceleration Voltage: 20 kV

### 6.1.1.2 Results

### (i) Antibody z10

### (a) Control Experiments

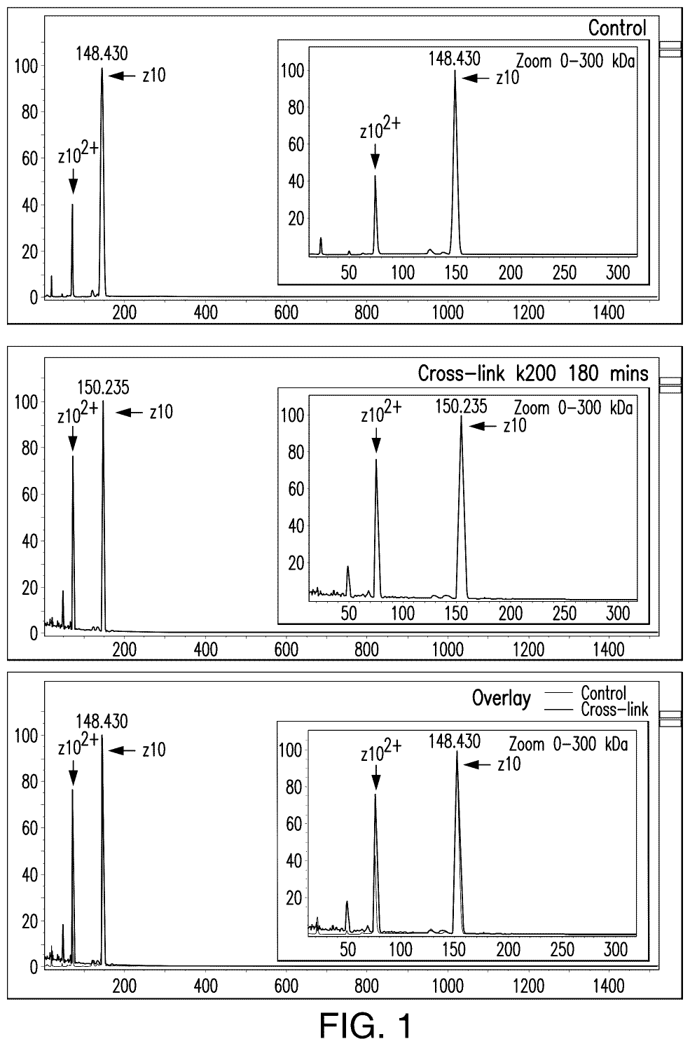

For these experiments, one main peak was detected for every dilution from 1 to 1/128 with MH+=148.430 kDa. (FIG. 1, Control, p 4) (Table 3).

TABLE 3

| Observed Molecular Weight (kDa) | |
|---|---|
| 148.430 | z10 |

### (b) Cross-Link Experiments

For these experiments, one main peak was detected for every dilution from 1 to 1/64 with MH+=150.235 kDa. (FIG. 1, Cross-link, p 4) (Table 4).

TABLE 4

| Observed Molecular Weight (kDa) | |
|---|---|
| 150.235 | z10 |

Using complex tracker software no non-covalent complexes were detected in the higher mass range (FIG. 1, Overlay, p 4).

(ii) rhMER_Fc

### (c) Control Experiments

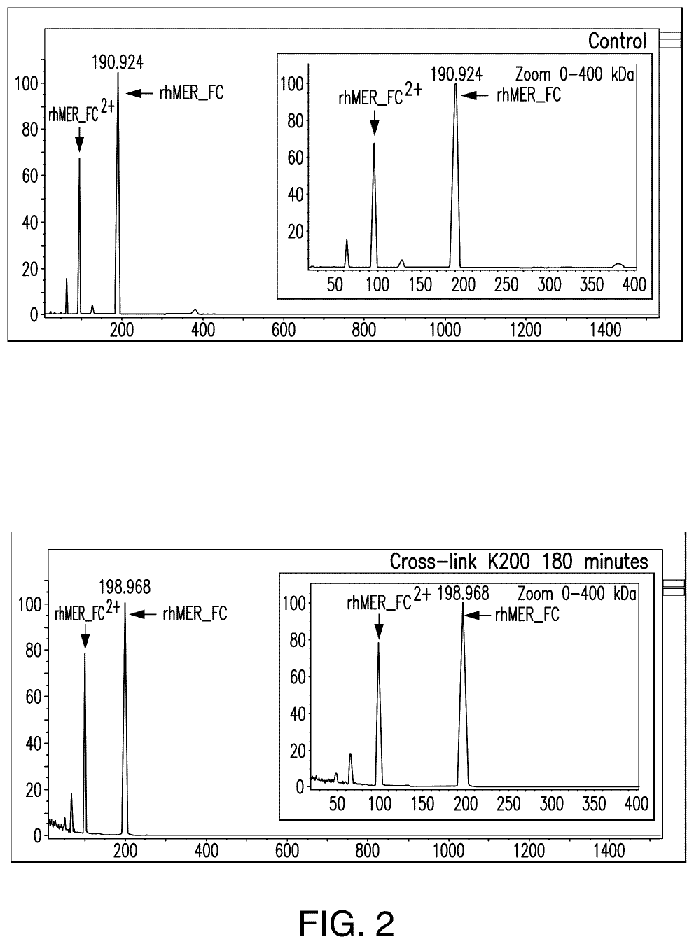

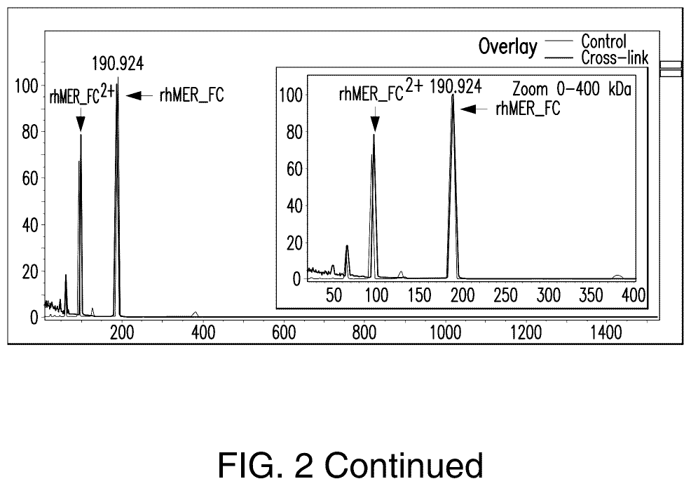

For these experiments, one main peak was detected for every dilution from 1 to 1/64 with MH+=190.924 kDa (FIG. 2, Control, p 5) (Table 5).

TABLE 5

| Observed Molecular Weight (kDa) | |
|---|---|
| 190.924 | rhMER_Fc |

### (d) Cross-Link Experiments

For these experiments, one main peak was detected for every dilution from 1 to 1/16 with MH+=198.968 kDa (FIG. 2, Cross-link, p 5) (Table 6).

TABLE 6

| Observed Molecular Weight (kDa) | |
|---|---|
| 198.968 | rhMER_Fc |

Using complex tracker software no non-covalent complexes were detected in the higher mass range (FIG. 2, Overlay, p 5).

### 6.1.1.3 Conclusion Aggregation Test

Using High-Mass MALDI mass spectrometry and chemical cross-linking, no non-covalent aggregates of z10 or multimers rhMER_Fc were detected.

### 6.1.2 Characterization of the z10/rhMER_Fc Complex

### 6.1.2.1 Materials and Methods

### (i) Instrumentation

For the characterization of z10/rhMER_Fc complex, the measurements were performed using an Autoflex II MALDI ToF mass spectrometer (Bruker) equipped with CovalX's HM4 interaction module. CovalX's interaction module contains a special detecting system designed to optimize detection up to 2 MDa with nano-molar sensitivity.

### (ii) Sample Preparation

### (a) Control Experiments

Mixture of z10/rhMER_Fc was prepared with the concentrations shown in Table 7:

TABLE 7

| | z10 | | rhMER_Fc | | z10/rhMER_Fc | |
|---|---|---|---|---|---|---|
| Mixtures | Volume | Conc. | Volume | Conc. | Volume | Conc. |
| z10/rhMER_Fc | 5 μl | 1.68 | 5 μl | 5.24 | 10 μl | 0.84 μM/ |
| | | μM | | μM | | 2.62 μM |

1 μl of the mixture obtained was mixed with 1 μl of a matrix composed of a re-crystallized sinapinic acid matrix (10 mg/ml) in acetonitrile/water (1:1, v/v), TFA 0.1% (K200 MALDI Kit). After mixing, 1 μl of each sample was spotted on the MALDI plate (SCOUT 384). After crystallization at room temperature, the plate was introduced in the MALDI mass spectrometer and analyzed immediately. The analysis was repeated in triplicate.

### (b) Cross-Link Experiments

The mixture prepared for the control experiment (9 μl left) was submitted to cross-linking using CovalX's K200 MALDI MS analysis kit. 9 μl of the mixture was mixed with 1 μl of K200 Stabilizer reagent (2 mg/ml) and incubated at room temperature. After the incubation time (360 minutes) the samples were prepared for MALDI analysis as for Control experiments. The samples were analyzed by High-Mass MALDI analysis immediately after crystallization.

(iii) High-Mass MALDI MS Analysis

The MALDI ToF MS analysis was performed using CovalX's HM4 interaction module with a standard nitrogen laser and focusing on different mass ranges from 0 to 1500 kDa.

For the analysis, the following parameters were applied:
Mass Spectrometer:
Linear and Positive mode
Ion Source 1: 20 kV
Ion Source 2: 17 kV
Lens: 12 kV
Pulse Ion Extraction: 400 ns
HM4:
Gain Voltage: 3.14 kV
Acceleration Voltage: 20 kV

To calibrate the instrument, an external calibration with clusters of Insulin, BSA and IgG was applied. For each sample, 3 spots were analyzed (300 laser shots per spots). The presented spectrum corresponds to the sum of 300 laser shots. The MS data were analyzed using CovalX's Complex Tracker analysis software version 2.0.

### 6.1.2.2 Results

(i) z10/rhMER_Fc

### (a) Control Experiments

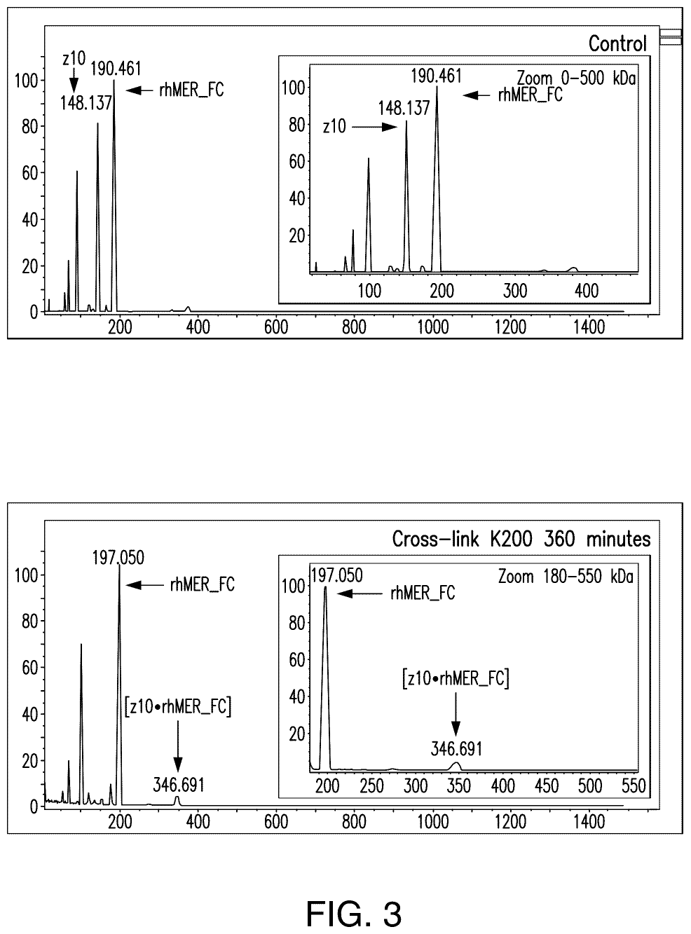

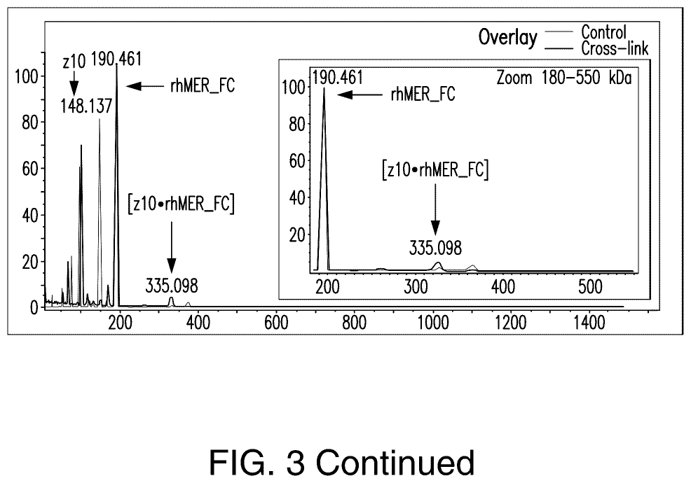

For this experiment, z10 and rhMER_Fc were detected with MH+=148.137 kDa and MH+=190.461 kDa, respectively (FIG. 3, Control, p 8) (Table 8).

TABLE 8

| Observed Molecular Weight (kDa) | |
|---|---|
| 148.137 | z10 |
| 190.461 | rhMER_Fc |

### (b) Cross-Link Experiments

The cross-linking experiment was completed after 360 minutes incubation time with the K200 reagent. After cross-linking, one additional peak was detected with MH+=346.691 kDa (FIG. 3, Cross-link, p 8) (Table 9).

TABLE 9

| Observed Molecular Weight (kDa) | |
|---|---|
| 346.691 | [z10 •rhMER_Fc] |

Using Complex Tracker software, the control and cross-link spectra were overlaid. One non-covalent protein complex with MH+=335.098 kDa was detected (FIG. 3, Overlay, p 8) (Table 10).

TABLE 10

| Observed Molecular Weight (kDa) | |
|---|---|
| 335.098 | [z10 •rhMER_Fc] |

### 6.1.3 Characterization and Peptide Mass Fingerprint of rhMER_Fc

In order to characterize rhMER_Fc the sample was subjected to trypsin, chymotrypsin, Asp-N, elastase and thermolysin proteolysis followed by nLC-LTQ-Orbitrap MS/MS analysis.

### 6.1.3.1 Materials and Methods

### (i) Instrumentation

For the characterization of rhMER_Fc, a nLC Ultimate 3000-RSLC system in line with a LTQ-Orbitrap mass spectrometer (Thermo Scientific) was used.

### (ii) Sample Preparation:

### (a) Reduction Alkylation

10 μL of rhMER_Fc (2.63 μM) were mixed with 1 μL of DSS d0/d12 (2 mg/mL;DMF) before 180 minutes incubation time at room temperature. After incubation, reaction was stopped by adding 1 μL of Ammonium Bicarbonate (20 mM final concentration) before 1 h incubation time at room temperature. Then, the solution was dried using a speedvac before H₂O 8 M urea suspension (10 μL). After mixing, 1 μl of DTT (500 mM) was added to the solution. The mixture was then incubated 1 hour at 37° C. After incubation, 1 μl of iodoacetamide (1 M) was added before 1 hour incubation time at room temperature, in a dark room. After incubation, 100 μl of the proteolytic buffer were added. The trypsin buffer contains 50 mM Ambic pH 8.5, 5% acetonitrile, the chymotrypsin buffer contains Tris HCl 100 mM, CaCL₂ 10 mM pH 7.8: The ASP-N buffer contains Phosphate buffer 50 mM pH 7.8; The elastase buffer contains Tris HCl 50 mM pH 8.0 and the thermolysin buffer contains Tris HCl 50 mM, CaCL₂ 0.5 mM pH 9.0.

### (b) Trypsin Proteolysis

100 μl of the reduced/alkylated rhMER_Fc were mixed with 1 μl of trypsin (Roche Diagnostic) with the ratio 1/100. The proteolytic mixture was incubated overnight at 37° C.

### (c) Chymotrypsin Proteolysis

100 μl of the reduced/alkylated rhMER_Fc were mixed with 0.5 μl of chymotrypsin (Roche Diagnostic) with the ratio 1/200. The proteolytic mixture was incubated overnight at 25° C.

### (d) ASP-N Proteolysis

100 μl of the reduced/alkylated rhMER_Fc were mixed with 0.5 μl of ASP-N(Roche Diagnostic) with the ratio 1/200. The proteolytic mixture was incubated overnight at 37° C.

### (e) Elastase Proteolysis

100 μl of the reduced/alkylated rhMER_Fc were mixed with 1 μl of elastase (Roche Diagnostic) with the ratio 1/100. The proteolytic mixture was incubated overnight at 37° C.

### (f) Thermolysin Proteolysis

100 μl of the reduced/alkylated rhMER_Fc were mixed with 2 μl of thermolysin (Roche Diagnostic) with a ratio 1/50. The proteolytic mixture was incubated overnight at 70° C.

After digestion formic acid 1% final was added to the solution.

(iii) Liquid Chromatography

After proteolysis, 10 μl of the peptide solution generated by proteolysis was loaded onto a nano-liquid chromatography system (Ultimate 3000-RSLC).

| | |
|---|---|
| A | 98/02/0.1 H2O/ACN/HCOOH v/v/v |
| B | 20/80/0.1 H2O/ACN/HCOOH v/v/v |
| gradient | 2-40% B in 38 minutes |
| injected volume | 10 μl |
| precolumn | 300-μm ID × 5-mm C18 PepMapTM |
| precolumn flow rate | 10 μl/min |
| column | 75-μm ID × 25-cm C18 PepMapRSLC |
| column flow rate | 300 nl/min |

### (iv) Mass Spectrometry: LTQ-Orbitrap MS Analysis

The LTQ-Orbitrap MS analysis has been performed with the following parameters:

| needle voltage | 1.8 | V |
|---|---|---|
| capillary voltage | 5 | V |
| μscan MS | 1 | |
| μscan MS² | 1 | |
| MS range m/z | 350-1700 | |
| MS/MS strategy | MS + 6 CID | |
| Min. signal required | MS/MS 500 | |
| Ion isolation window | 3 | m/z units |

| | |
|---|---|
| Normalized collision energy | 35% |
| Default charge state | 3 |
| Activation Q | 0.25 |
| Activation time | 30 |
| Dynamic exclusion | ON |
| Dynamic exclusion params | RC 1, RD 30s, ED 30s |
| Charge state screening | Not applicable |
| Charge state rejection | Not Applicable |
| Charge state reject. Params | +1 and unassigned rejected |

### (v) Data Analysis

### Input Data

Enzyme: Trypsin
Max. Missed Cleavage Sites: 2
Min. Peptide Length: 6
Max. Peptide Length: 144

### Scoring Options

Max. Delta On: 0.05

### Tolerances

Precursor Mass Tolerance: 10 ppm
Fragment Mass Tolerance: 0.6 Da
Use Average Precursor Mass: False
Use Average Fragment Mass: False

### Spectrum Matching

Use Neutral Loss a Ions: False
Use Neutral Loss b Ions: True
Use Neutral Loss y Ions: True
Use Flanking Ions: True

### Modifications

Max.Equal Modifications Per Peptide: 3
Dynamic Modification: Oxidation/+15.995 Da[M]
Static Modification: Carbamidomethyl/+57.021 Da [C]

### General Settings

Precursor Selection: Use MS1 Precursor
Min. Precursor Mass: 350 Da
Max. Precursor Mass: 5000 Da
Total Intensity Threshold: 0
Minimum Peak Count: 1

### Scan Event Filters

Mass Analyzer: Any
MS Order: Is MS2
Activation type: Any
Min. Collision Energy: 0
Max. Collision Energy: 1000
Scan Type: Is Full

### Peak Filters

S/N Threshold (FT-only): 1.5

### Replacements for Unrecognized Properties

Unrecognized Charge Replacements: Automatic
Unrecognized Mass Analyzer Replacements: ITMS
Unrecognized MS Order Replacements: MS2
Unrecognized Activation Replacements: HCD
Unrecognized Polarity Replacements: +

### Database

Name: 2018_12_BanqueProtCov_Keratin_RGENIX.fasta
Description: BanqueprotCov+keratin bank.+Sequence client.
Format:Fasta; indexed

TABLE 11

| | Species | KT | Total |
|---|---|---|---|
| Direct | 4308 | 334 | 4642 |
| Reversed | 0 | 0 | 0 |
| Total | 4308 | 334 | 4642 |

### 6.1.3.2 Results

### (a) Trypsin Proteolysis

Peptides identified in the sequence of rhMER_Fc, covering 54.22% of the sequence are shown in Table 12:

TABLE 12
Identified peptides of
rhMER_Fc after trypsin proteolysis.

| | | | SEQ |
|---|---|---|---|
| | | Position | ID |
| Sequence | Modifications | Peptide* | NO. |
| HTVGHIILSEHK | | 73-84 | 2 |
| HTVGHIILSEHKGVK | | 73-87 | 3 |
| FNCSISVPNIYQDTT | intra-protein xl | 88-110 | 4 |
| ISWWKDGK | | | |
| SDNGSYIcK | C8 | 143-151 | 5 |
| | (Carbamidomethyl) | | |
| mKINNEEIVSDPIYI | M1(Oxidation) | 152-176 | 6 |
| EVQGLPHFTK | | | |
| INNEEIVSDPIYIEV | | 154-176 | 7 |
| QGLPHFTK | | | |
| QPESMNVTR | | 177-185 | 8 |
| SPSVLTVPGLTEMAV | intra-protein xl | 220-256 | 9 |
| FSCEAHNDKGLTVSK | | | |
| GVQINIK | | | |
| AIPSPPTEVSIR | | 257-268 | 10 |
| NSTAHSILISWVPGF | | 269-290 | 11 |
| DGYSPFR | | | |
| NcSIQVK | C2 | 291-297 | 12 |
| | (Carbamidomethyl) | | |
| EADPLSNGSVMIFNT | monolink | 298-322 | 13 |
| SALPHLYQIK | | | |
| WmKPPTK | M2(Oxidation) | 380-386 | 14 |
| WmKPPTKQQDGELVG | M2(Oxidation) | 380-396 | 15 |
| YR | | | |
| QQDGELVGYR | | 387-396 | 16 |
| ISHVWQSAGISK | | 397-408 | 17 |
| ELLEEVGQNGSR | | 409-420 | 18 |
| ARISVQVHNATcTVR | C12 | 421-435 | 19 |
| | (Carbamidomethyl) | | |
| ISVQVHNATcTVR | C10 | 423-435 | 20 |
| | (Carbamidomethyl) | | |
| IAAVTRGGVGPFSDP | | 436-452 | 21 |
| VK | | | |
| GGVGPFSDPVK | | 442-452 | 22 |

*The MERTK sequence contained in rhMER_Fc consists of amino acid numbers 26-999 of SEQ ID NO: 1. The numbering of the peptide positions in Table 12 starts with the first MERTK amino acid in rhMER_Fc, and thus the given position plus 25 corresponds to the amino acid position in SEQ ID NO: 1. Thus, for example, position peptide 73-84 in Table 12 is amino acid numbers 98-109 of SEQ ID NO: 1.

### (b) Chymotrypsin Proteolysis

Peptides identified in the sequence of rhMER_Fc, covering 5.06% of the sequence are shown in Table 13:

TABLE 13
Identified peptides of
rhMER_Fc after Chymotrypsin proteolysis.

| | | | SEQ |
|---|---|---|---|
| | Modifi- | Position | ID |
| Sequence | cations | Peptide* | NO. |
| SPFRNCSIQVKEADPLSNGSVMIF | monolink | 287-310 | 23 |

*The MERTK sequence contained in rhMER_Fc consists of amino acid numbers 26-999 of SEQ ID NO: 1. The numbering of the peptide positions in Table 13 starts with the first MERTK amino acid in rhMER_Fc, and thus the given position plus 25 corresponds to the amino acid position in SEQ ID NO: 1.

### (c) ASP-N Proteolysis

Peptides identified in the sequence of rhMER_Fc, covering 12.24% of the sequence are shown in Table 14:

TABLE 14
Identified peptides of
rhMER_Fc after ASP-N proteolysis.

| | | | SEQ |
|---|---|---|---|
| | Modifi- | Position | ID |
| Sequence | cations | Peptide* | NO. |
| REEAKPYPLFPGPFPGSLQT | | 1-20 | 24 |
| DIRWmKPPTKQQ | M5(Oxidation) | 377-388 | 25 |
| DPVKIFIPAHGWV | | 449-461 | 26 |
| DYAPSSTPAPGNA | | 462-474 | 27 |

*The MERTK sequence contained in rhMER_Fc consists of amino acid numbers 26-999 of SEQ ID NO: 1. The numbering of the peptide positions Table 14 starts with the first MERTK amino acid in rhMER_Fc, and thus the given position plus 25 corresponds to the amino acid position in SEQ ID NO: 1..

### (d) Elastase Proteolysis

Peptides identified in the sequence rhMER_Fc, covering 88.82% of the sequence are shown in Table 15:

TABLE 15
Identified peptides of
rhMER_Fc after Elastase proteolysis.

| | | | SEQ |
|---|---|---|---|
| | Modifi- | Position | ID |
| Sequence | cations | Peptide* | NO. |
| REEAKPYPL | | 1-9 | 28 |
| REEAKPYPLFPGPFP | | 1-18 | 29 |
| GSL | | | |
| FPGPFPGSLQTDHTP | | 10-25 | 30 |
| L | | | |
| GYQPA | intra-protein xl | 33-37 | 31 |
| LmFSPTQPGRPHTGN | M2(Oxidation) | 38-53 | 32 |
| V | | | |
| LMFSPTQPGRPHTGN | | 38-54 | 33 |
| VA | | | |
| IPQVTSV | intra-protein xl | 55-61 | 34 |
| ESKPLPPLA | | 62-70 | 35 |
| ESKPLPPLAFKHTV | | 62-75 | 36 |
| FKHTVGHII | | 71-79 | 37 |
| ILSEHKGV | | 79-86 | 38 |
| KFNCSIS | | 87-93 | 39 |
| VPNIYQDTTI | | 94-103 | 40 |
| SWWKDGKEL | | 104-112 | 41 |
| GAHHA | | 114-118 | 42 |
| ITQFYPDDEVTA | | 119-130 | 43 |
| TQFYPDDEVTAI | | 120-131 | 44 |
| QRSDNGSYI | | 141-149 | 45 |
| CKMKINNEEIV | | 150-160 | 46 |
| NNEEIVSDPI | | 155-164 | 47 |
| DPIYIEV | | 162-168 | 48 |
| QGLPHFTKQPESMNV | | 169-183 | 49 |
| TRNTAFNLTCQA | | 184-195 | 50 |
| TCQAVGPPEPV | | 192-202 | 51 |
| SRVNEQPEKSPS | | 211-222 | 52 |
| NEQPEKSPSVL | intra-protein xl | 214-224 | 53 |
| TVPGLTEMAV | | 225-234 | 54 |
| VFScEAHNDKGL | C4 | 234-245 | 55 |
| | (Carbamidomethyl) | | |
| FScEAHNDKGL | C3 | 235-245 | 56 |
| | (Carbamidomethyl) | | |
| FScEAHNDKGLTV | C3 | 235-247 | 57 |
| | (Carbamidomethyl) | | |
| HNDKGLTV | | 240-247 | 58 |
| QINIKA | intra-protein xl | 252-257 | 59 |
| NIKAIPSPPTEV | | 254-265 | 60 |
| SIRNSTAHSI | | 266-275 | 61 |
| SWVPG | intra-protein xl | 278-282 | 62 |
| FDGYSPFRNCS | | 283-293 | 63 |
| YSPFRNCSIQV | | 286-296 | 64 |
| QVKEA | | 295-299 | 65 |
| FNTSALPHL | | 310-318 | 66 |
| LPHLYQI | | 315-321 | 67 |
| LPHLYQIKQL | | 315-324 | 68 |
| KQLQAL | | 322-327 | 69 |
| ANYSI | intra-protein xl | 328-332 | 70 |
| VSCMNEI | | 334-340 | 71 |
| ScMNEIGWSA | C2 | 335-344 | 72 |
| | (Carbamidomethyl) | | |
| SPWILA | | 346-351 | 73 |
| TTEGAPSV | | 353-360 | 74 |
| APLNVTV | | 361-367 | 75 |
| TVFLNES | | 366-372 | 76 |
| NESSDNV | | 370-376 | 77 |
| DIRWMKPPTKQQDGE | | 377-393 | 78 |
| LV | | | |
| RWMKPPTKQQDGEL | | 379-392 | 79 |
| RWmKPPTKQQDGELV | M3(Oxidation) | 379-393 | 80 |
| RWMKPPTKQQDGELV | | 379-397 | 81 |
| GYRI | | | |
| YRISHV | intra-protein xl | 395-400 | 82 |
| WQSAG | | 401-405 | 83 |
| ISKEL | | 406-410 | 84 |
| SKELLEEV | | 407-414 | 85 |
| SRARI | | 419-423 | 86 |
| RARIS | | 420-424 | 87 |
| QVHNATCTV | | 426-434 | 88 |
| HNATCTVRI | | 428-436 | 89 |
| VTRGGVGPFSDPV | | 439-451 | 90 |
| TRGGVGPFSDPV | | 440-451 | 91 |
| DPVKIFIPA | | 449-457 | 92 |
| HGWVDYAPS | | 458-466 | 93 |
| DYAPSSTPAPG | intra-protein xl | 462-472 | 94 |

*The MERTK sequence contained in rhMER_Fc consists of amino acid numbers 26-999 of SEQ ID NO: 1. The numbering of the peptide positions in Table 15 starts with the first MERTK amino acid in rhMER_Fc, and thus the given position plus 25 corresponds to the amino acid position in SEQ ID NO: 1.

### (e) Thermolysin Proteolysis

Peptides identified in the sequence of rhMER_Fc, covering 87.13% of the sequence are shown in Table 16:

TABLE 16
Identified peptides of
rhMER_Fc after Thermolysin proteolysis.

| | | | SEQ |
|---|---|---|---|
| | Modifi- | Position | ID |
| Sequence | cations | Peptide* | NO. |
| REEAKPYP | | 1-8 | 95 |
| AKPYPLFPGPFPGS | | 4-17 | 96 |
| LFPGPFPGSLQTDHTPL | | 9-25 | 97 |
| LSLPHASGYQPA | | 26-37 | 98 |
| LMFSPTQPGRPHTGN | | 38-52 | 99 |
| MFSPTQPGRPHTGN | | 39-52 | 100 |
| FSPTQPGRPHTGN | | 40-52 | 101 |
| FSPTQPGRPHTGNVAIPQ | | 40-57 | 102 |
| VTSVESKPLPP | | 58-68 | 103 |
| VESKPLPP | | 61-68 | 104 |
| AFKHTVGH | | 70-77 | 105 |
| IILSEHKG | | 78-85 | 106 |
| LSEHKGVKFNCS | | 80-91 | 107 |
| VKFNCSISVPN | | 86-96 | 108 |
| IYQDTTISWWKDGKE | | 97-111 | 109 |
| IYQDTTISWWKDGKEL | | 97-112 | 110 |
| ISWWKDGKE | | 103-111 | 111 |
| AITQFYPDDE | intra-protein xl | 118-127 | 112 |
| ITQFYPDDEVT | | 119-129 | 113 |
| FSITSVQRSDNGSY | | 135-148 | 114 |
| IcKMKINNEE | C2 | 149-158 | 115 |
| | (Carbamidomethyl) | | |
| IVSDPIY | | 159-165 | 116 |
| IEVQGLPHFTKQPES | | 166-180 | 117 |
| FTKQPESMN | | 174-182 | 118 |
| MNVTRNTA | | 181-188 | 119 |
| FNLTCQA | | 189-195 | 120 |
| IFWVQNSSR | | 204-212 | 121 |
| VNEQPEKSPSVLTVPG | | 213-228 | 122 |
| LTVPGLTEM | | 224-232 | 123 |
| VFScEAHNDKG | C4 | 234-244 | 124 |
| | (Carbamidomethyl) | | |
| FScEAHNDKG | C3 | 235-244 | 125 |
| | (Carbamidomethyl) | | |
| LTVSKGVQ | | 245-252 | 126 |
| INIKAIPSPPTE | | 253-264 | 127 |
| IKAIPSPPTEVS | | 255-266 | 128 |
| AIPSPPTEVSIRNST | | 257-271 | 129 |
| IRNSTAHS | | 267-274 | 130 |
| ILISWVPG | | 275-282 | 131 |
| LISWVPGFDGYSP | | 276-288 | 132 |
| FDGYSPFRNCSIQ | | 283-295 | 133 |
| ALPHLYQIKQ | | 314-323 | 134 |
| LYQIKQLQ | | 318-325 | 135 |
| IKQLQA | | 321-326 | 136 |
| LANYSIG | | 327-333 | 137 |
| VSCMNE | | 334-339 | 138 |
| LASTTEGAPS | | 350-359 | 139 |
| LNVTV | | 363-367 | 140 |
| VDIRWMKPPTKQQDGE | intra-protein xl | 376-391 | 141 |
| MKPPTKQQDGE | | 381-391 | 142 |
| MKPPTKQQDGEL | | 381-392 | 143 |
| mKPPTKQQDGELVGYR | MI(Oxidation) | 381-396 | 144 |
| LVGYRISH | | 392-399 | 145 |
| VGYRISH | | 393-399 | 146 |
| ISHVWQSAG | | 397-405 | 147 |
| ISKEL | | 406-410 | 148 |
| LEEVGQNGSRAR | | 411-422 | 149 |
| VGQNGSRARIS | | 414-424 | 150 |
| VQVHN | | 425-429 | 151 |
| ATCTVRI | | 430-436 | 152 |
| AVTRGGVGP | | 438-446 | 153 |
| FSDPVKI | | 447-453 | 154 |
| FIPAHGWVDYAPSSTPAP | intra-protein xl | 454-473 | 155 |
| GN | | | |

*The MERTK sequence contained in rhMER_Fc consists of amino acid numbers 26-999 of SEQ ID NO: 1. The numbering of the peptide positions in Table 16 starts with the first MERTK amino acid in rhMER_Fc, and thus the given position plus 25 corresponds to the amino acid position in SEQ ID NO: 1..

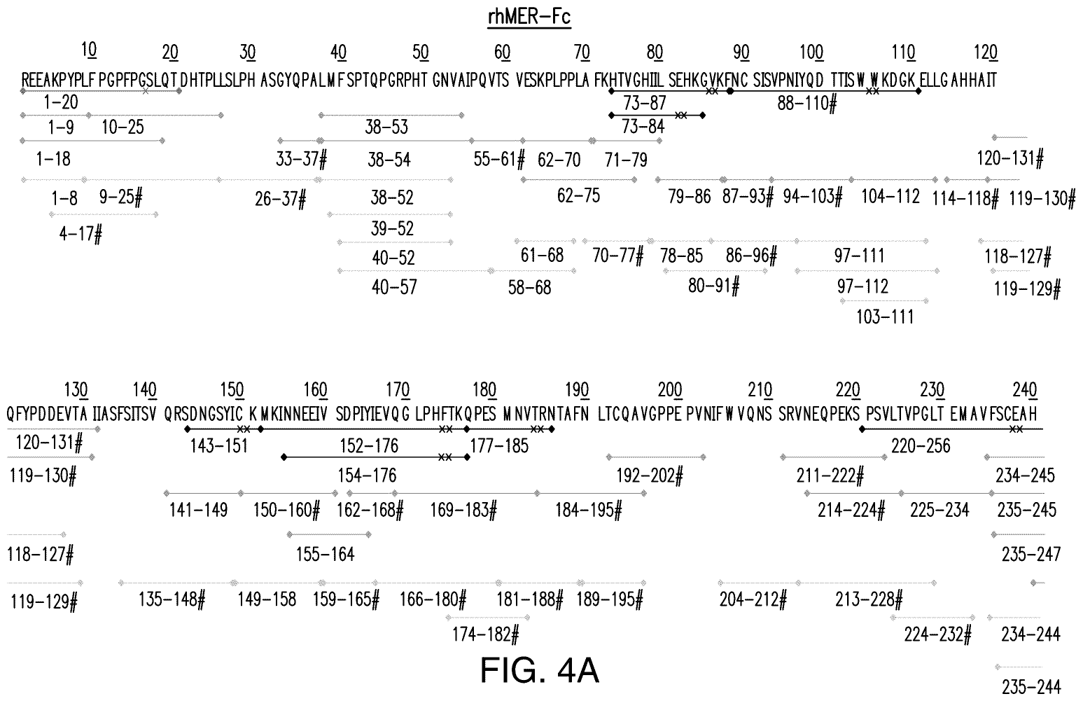

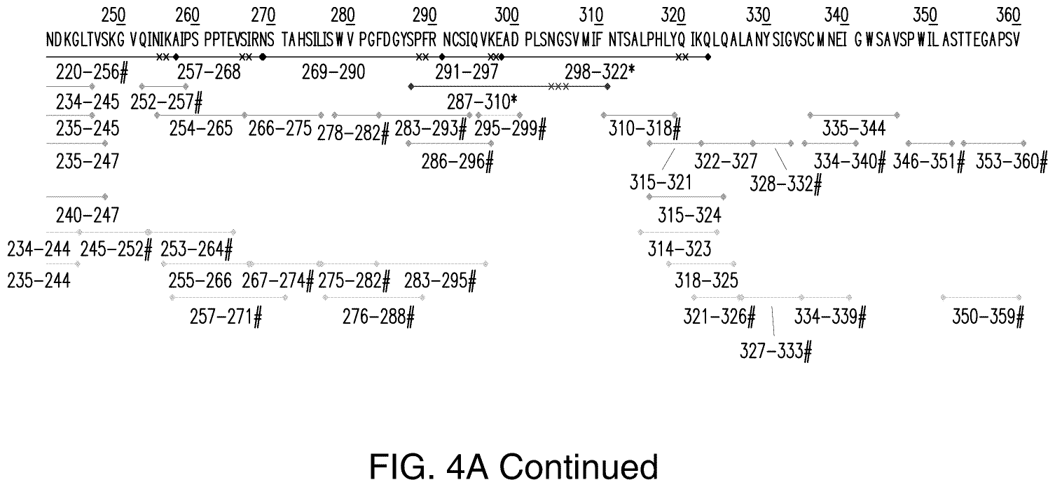

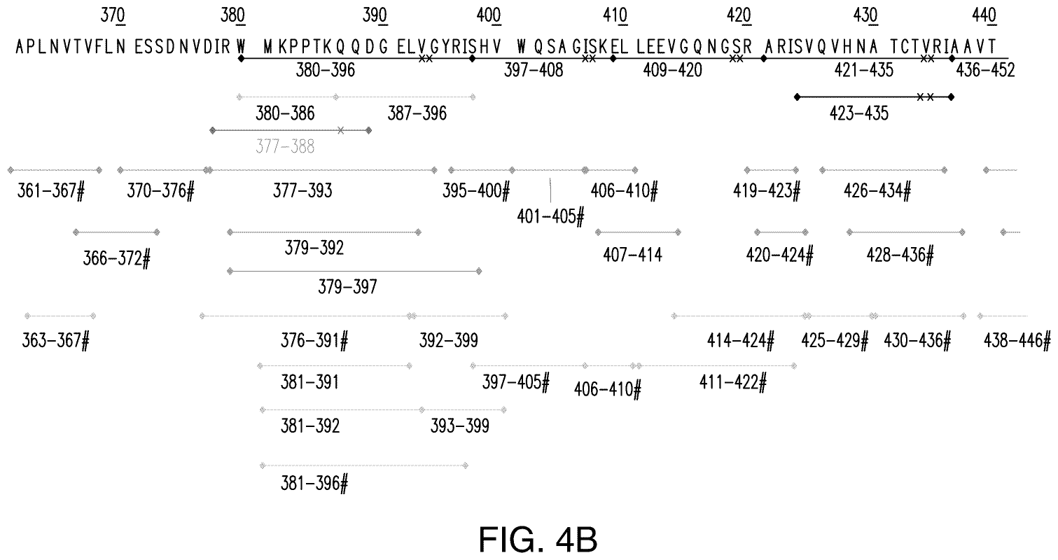

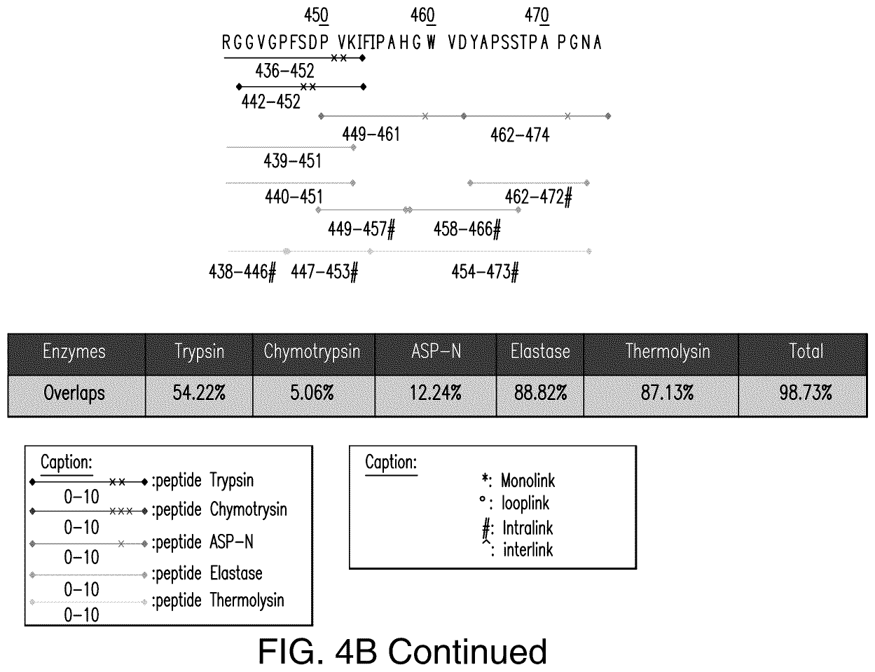

Based on the results obtained, overlap mapping of the trypsin, chymotrypsin, ASP-N, elastase and thermolysin peptides were designed (FIG. 4). Combining the peptides of Trypsin, Chymotrypsin, ASP-N, Elastase and Thermolysin proteolysis, 98.73% of the sequence is covered.

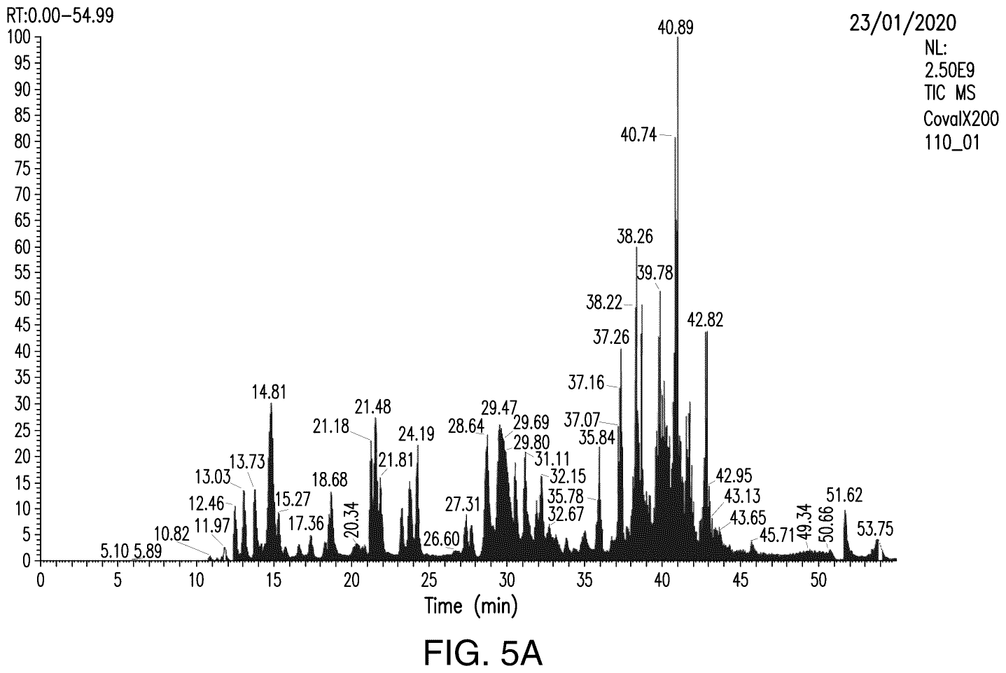

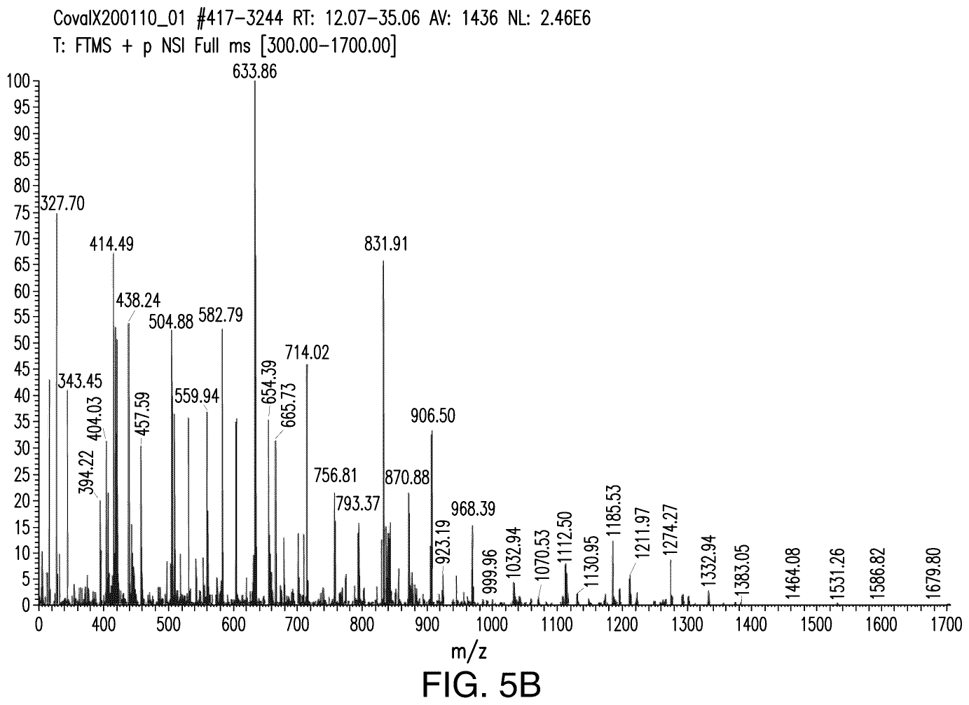

The nLC chromatogram and the total sum of the ions detected by the LTQ-Orbitrap for trypsin digest of rhMER_Fc are presented in FIG. 5.

### 6.1.4 Characterization of the Molecular Interface

In order to determine the epitope of z10/rhMER_Fc complexes with high resolution, the protein complexes were incubated with deuterated cross-linkers and subjected to multi-enzymatic cleavage. After enrichment of the cross-linked peptides, the samples were analyzed by high resolution mass spectrometry (nLC-LTQ-Orbitrap MS) and the data generated were analyzed using XQuest and Stavrox software.

### 6.1.4.1 Materials and Methods

### (i) Instrumentation

For this analysis, nLC chromatography in combination with LTQ-Orbitrap mass spectrometry have been used as described in sections 6.1.1.1(c) and 6.1.1.1(d).

### (ii) Sample Preparation:

Mixture of z10/rhMER_Fc was prepared with the concentrations shown in Table 17:

TABLE 17

| | rhMER_Fc | | z10 | | z10/rhMER_Fc | |
|---|---|---|---|---|---|---|
| Mixtures | Volume | Conc. | Volume | Conc. | Volume | Conc. |
| z10/rhMER_Fc | 10 μl | 1.68 | 10 μl | 5.24 | 20 μl | 0.84 μM/ |
| | | μM | | μM | | 2.62 μM |

### (a) Reduction Alkylation

20 μL of the z10/rhMER_Fc mixtures prepared were mixed with 2 μL of DSS d0/d12 (2 mg/mL;DMF) before 180 minutes incubation time at room temperature. After incubation, reaction was stopped by adding 1 μL of Ammonium Bicarbonate (20 mM final concentration) before 1 h incubation time at room temperature. Then, the solution was dried using a speedvac before H₂O 8M urea suspension (20 μL). After mixing, 2 μl of DTT (500 mM) were added to the solution. The mixture was then incubated 1 hour at 37° C. After incubation, 2 μl of iodoacetamide (1 M) were added before 1 hour incubation time at room temperature, in a dark room. After incubation, 80 μl of the proteolytic buffer were added. The trypsin buffer contains 50 mM Ambic pH 8.5, 5% acetonitrile; The Chymotrypsin buffer contains Tris HCl 100 mM, CaCl₂) 10 mM pH 7.8; The ASP-N buffer contains Phosphate buffer 50 mM pH 7.8; The elastase buffer contains Tris HCl 50 mM pH 8.0 and the thermolysin buffer contains Tris HCl 50 mM, CaCl₂) 0.5 mM pH 9.0.

### (b) Trypsin Proteolysis

100 μl of the reduced/alkylated z10/rhMER_Fc mixtures were mixed with 2.5 μl of trypsin (Roche Diagnostic) with the ratio 1/100. The proteolytic mixtures were incubated overnight at 37° C.

### (c) Chymotrypsin Proteolysis

100 μl of the reduced/alkylated z10/rhMER_Fc mixtures were mixed with 1.25 μl of chymotrypsin (Roche Diagnostic) with the ratio 1/200. The proteolytic mixtures were incubated overnight at 25° C.

### (d) ASP-N Proteolysis

100 μl of the reduced/alkylated z10/rhMER_Fc mixtures were mixed with 1.25 μl of ASP-N(Roche Diagnostic) with the ratio 1/200. The proteolytic mixtures were incubated overnight at 37° C.

### (e) Elastase Proteolysis

100 μl of the reduced/alkylated z10/rhMER_Fc mixtures were mixed with 2.5 μl of elastase (Roche Diagnostic) with the ratio 1/100. The proteolytic mixtures were incubated overnight at 37° C.

### (f) Thermolysin Proteolysis

100 μl of the reduced/alkylated z10/rhMER_Fc mixtures were mixed with 5.0 μl of thermolysin (Roche Diagnostic) with a ratio 1/50. The proteolytic mixtures were incubated overnight at 70° C.

After digestion formic acid 1% final was added to the solution

(iii) Data Analysis

The cross-linked peptides were analyzed using Xquest version 2.0 and Stavrox 3.6. software.

### 6.1.4.2 Results

After Trypsin, Chymotrypsin, ASP-N, Elastase and Thermolysin proteolysis of the protein complex z10/rhMER_Fc with deuterated d0d12, the nLC-orbitrap MS/MS analysis detected 19 cross-linked peptides between rhMER_Fc and the antibody z10.

The sequences and positions of cross-links are presented in Table 18, below.

TABLE 18
z10-Trypsin, Chymotrypsin, ASP-N, Elastase and Thermolysin
Interlink between z10 complementarity determining regions and rhMER_Fc

| | | Protein | Protein | Sequence | Sequence | | | | Identified |
|---|---|---|---|---|---|---|---|---|---|
| Sequence | Enzyme | 1 | 2 | Protein 1 | Protein 2* | XL Type | nAA1 | nAA2* | on StavroX |
| TWYQQKPGKAPKL-STTEG-a1- | Elastase | z10_VL | RhMer_Fc | 34-46 | 352-356 | inter- | 34 | 354 | YES |
| b3 | | | | (SEQ ID | (SEQ ID | protein xl | | | |
| | | | | NO. 156) | NO. 157) | | | | |
| EWMGWINTYTGEPTYA- | AspN | z10_VH | RhMer_Fc | 46-61 | 355-370 | inter- | 59 | 359 | YES |
| EGAPSVAPLNVTVFLN-a14-b5 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 54 | 359 | YES |
| EGAPSVAPLNVTVFLN-a9-b5 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 53 | 359 | YES |
| EGAPSVAPLNVTVFLN-a8-b5 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 60 | 359 | YES |
| EGAPSVAPLNVTVFLN-a15-b5 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 55 | 359 | YES |
| EGAPSVAPLNVTVFLN-a10-b5 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 53 | 366 | YES |
| EGAPSVAPLNVTVFLN-a8-b12 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 54 | 366 | YES |
| EGAPSVAPLNVTVFLN--a9-b12 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 59 | 366 | YES |
| EGAPSVAPLNVTVFLN-a14-b12 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 60 | 366 | YES |
| EGAPSVAPLNVTVFLN-a15-b12 | | | | (SEQ ID | (SEQ ID | protein xl | | | |
| | | | | NO. 158) | NO. 159) | | | | |
| EWMGWINTYTGEPTYA- | AspN | z10_VH | RhMer_Fc | 46-61 | 355-370 | inter- | 55 | 366 | YES |
| EGAPSVAPLNVTVFLN-a10-b12 | | | | (SEQ ID | (SEQ ID | protein xl | | | |
| | | | | NO. 158) | NO. 159) | | | | |
| CARKSTVVSRY- | Chymo- | z10_VH | RhMer_Fc | 96-106 | 364-380 | inter- | 105 | 379 | YES |
| NVTVFLNESSDNVDIRW-a10- | trypsin | | | (SEQ ID | (SEQ ID | protein xl | | | |
| b16 | | | | NO. 160) | NO. 161) | | | | |
| DFATYYCQQYRSYPLTFGQGT | AspN | z10_VL | RhMer_Fc | 82-107 | 371-388 | inter- | 92 | 379 | YES |
| KLEIK-ESSDNVDIRWMKPPTKQQ- | | | | (SEQ ID | (SEQ ID | protein xl | | | |
| a11-b9 | | | | NO. 162) | NO. 163) | | | | |
| SGYTFTNYG- | Elastase | z10_VH | RhMer_Fc | 25-33 | 374-390 | inter- | 30 | 385 | YES |
| DNVDIRWMKPPTKQQDG-a6- | | | | (SEQ ID | (SEQ ID | protein xl | | | |
| b12 | | | | NO. 164) | NO. 165) | | | | |
| RSDDMAVYYCARKSTVVSRY- | Chymo- | z10_VH | RhMer_Fc | 87-106 | 381-395 | inter- | 104 | 385 | YES |
| MKPPTKQQDGELVGY-a18-b5 | trypsin | | | (SEQ ID | (SEQ ID | protein xl | | | |
| | | | | NO. 166) | NO. 167) | | | | |
| CQQYRSYPL- | Chymo- | z10_VL | RhMer_Fc | 88-96 | 381-392 | inter- | 92 | 386 | YES |
| MKPPTKQQDGEL-a5-b6 | trypsin | | | (SEQ ID | (SEQ ID | protein xl | | | |
| | | | | NO. 168) | NO. 169) | | | | |
| INTYTGEPTYADDFKGR- | Thermo- | z10_VH | RhMer_Fc | 51-67 | 381-396 | inter- | 53 | 395 | YES |
| MKPPTKQQDGELVGYR-a3-b15 | lysin | | | (SEQ ID | (SEQ ID | protein xl | | | |
| | | | | NO. 170) | NO. 171) | | | | |
| TNYGMNWVRQAPGQGL- | Chymo- | z10_VH | RhMer_Fc | 30-45 | 381-401 | inter- | 32 | 395 | YES |
| MKPPTKQQDGELVGYRISHVW- | trypsin | | | (SEQ ID | (SEQ ID | protein xl | | | |
| a3-b15 | | | | NO. 172) | NO. 173) | | | | |
| WASTRHTGVPDRFSGSGSGTDF- | Chymo- | z10_VL | RhMer_Fc | 50-71 | 396-411 | inter- | 56 | 398 | YES |
| RISHVWQSAGISKELL-a7-b3 | trypsin | | | (SEQ ID | (SEQ ID | protein xl | | | |
| | | | | NO. 174) | NO. 175) | | | | |

*The MERTK sequence contained in rhMER_Fc consists of amino acid numbers 26-999 of SEQ ID NO: 1. The numbering of the peptide positions in Table 18 starts with the first MERTK amino acid in rhMER_Fc, and thus the given position plus 25 corresponds to the amino acid position in SEQ ID NO: 1..

### 6.1.4.3 Conclusion Epitope Mapping

Using chemical cross-linking, High-Mass MALDI mass spectrometry and nLC-Orbitrap mass spectrometry the molecular interface between rhMER_Fc and the antibody z10 was characterized.

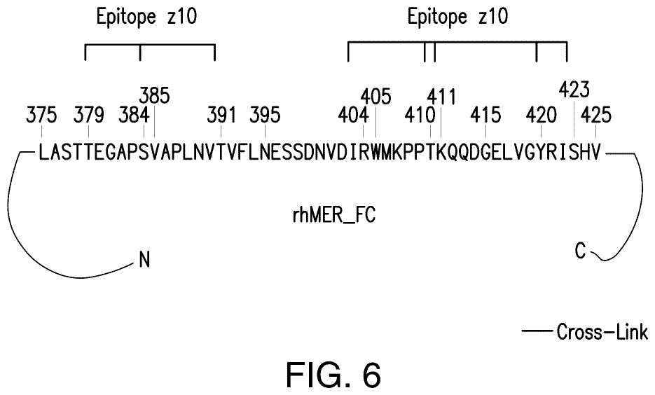

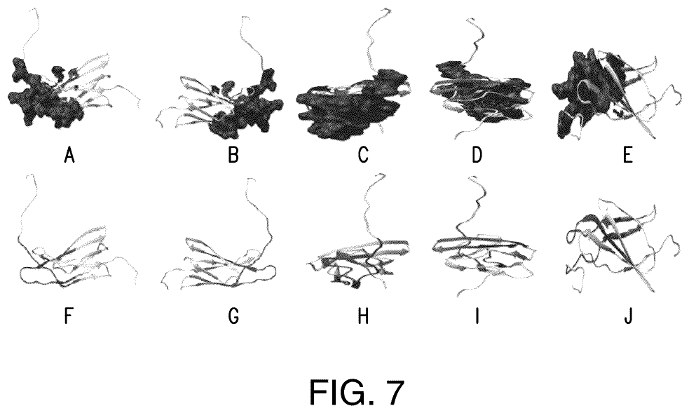

Our analysis indicates that the interaction includes the following amino acids on rhMER_Fc: 354, 359, 366; 379, 385, 386, 395, 398, which correspond to amino acid numbers 379, 384, 391, 404, 410, 411, 420, and 423, respectively, of SEQ ID NO:1. These results are illustrated in FIGS. 6 and 7.

The foregoing is not to be limited in scope by the specific embodiments described herein. Indeed, various modifications of the antibodies and methods provided herein and their equivalents, in addition to those described herein will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A polypeptide comprising a contiguous amino acid sequence of human MERTK (SEQ ID NO: 1) or a variant of said contiguous amino acid sequence, wherein the contiguous amino acid sequence comprises amino acids numbers 379-423 of the human MERTK sequence (SEQ ID NO: 1) and not more than 400 contiguous amino acids of the human MERTK sequence (SEQ ID NO: 1), and wherein the variant (a) has one or more amino acid substitutions, insertions, or deletions in the contiguous amino acid sequence relative to SEQ ID NO: 1, and (b) has at least 90% sequence identity over each of amino acid numbers 379-391 and 404-423 of SEQ ID NO: 1, or has only conservative substitutions relative to amino acid numbers 379-391 and 404-423 of SEQ ID NO: 1 and not more than two conservative substitutions relative to amino acid numbers 379-391 and 404-423 of SEQ ID NO: 1.

2. The polypeptide of claim 1, wherein the contiguous amino acid sequence comprises amino acid numbers 286-484 of SEQ ID NO: 1

3. The polypeptide of claim 1, which comprises the contiguous amino acid sequence.

4. The polypeptide of claim 2, which comprises the contiguous amino acid sequence.

5. The polypeptide of claim 1, which comprises the variant.

6. The polypeptide of claim 5, wherein the variant has at least 90% sequence identity over each of amino acid numbers 379-391 and 404-423 of SEQ ID NO: 1.

7. The polypeptide of claim 5, wherein the variant has only conservative substitutions relative to amino acid numbers 379-391 and 404-423 of SEQ ID NO: 1 and has not more than two conservative substitutions relative to amino acid numbers 379-391 and 404-423 of SEQ ID NO: 1.

8. The polypeptide of claim 5, wherein the variant has at least 90% sequence identity over amino acid numbers 286-484 of SEQ ID NO: 1.

9. The polypeptide of claim 5, wherein the variant has only conservative substitutions relative to amino acid numbers 286-484 of SEQ ID NO: 1.

10. The polypeptide of any one of claims 1-9, wherein the contiguous amino acid sequence comprises not more than 300 contiguous amino acids of the human MERTK sequence (SEQ ID NO: 1).

11. The polypeptide of any one of claims 1-9, wherein the contiguous amino acid sequence comprises not more than 200 contiguous amino acids of the human MERTK sequence (SEQ ID NO: 1).

12. The polypeptide of any one of claims 1, 3, and 5-7, wherein the contiguous amino acid sequence comprises not more than 100 contiguous amino acids of the human MERTK sequence (SEQ ID NO: 1).

13. The polypeptide of any one of claims 1, 3, and 5-7, wherein the contiguous amino acid sequence comprises not more than 50 contiguous amino acids of the human MERTK sequence (SEQ ID NO: 1).

14. The polypeptide of any one of claims 1-13, which consists of the contiguous amino acid sequence.

15. The polypeptide of any one of claims 1-13 wherein the polypeptide is a fusion protein comprising the contiguous amino acid sequence linked to a second amino acid sequence.

16. The polypeptide of claim 15 wherein the second amino acid sequence comprises the amino acid sequence of an adjuvant.

17. The polypeptide of claim 16 wherein the adjuvant is keyhole limpet hemocyanin.

18. The polypeptide of claim 15 wherein the second amino acid sequence comprises a tag or label.

19. The polypeptide of any one of claims 1-18, wherein the polypeptide is in lyophilized form.

20. A conjugate comprising the polypeptide of any one of claims 1-18 bound to a molecule.

21. The conjugate of claim 20 wherein the molecule is an adjuvant.

22. The conjugate of claim 20 or 21 wherein the molecule is covalently bound to the polypeptide.

23. The conjugate of any one of claims 20-22, wherein the conjugate is in lyophilized form.

24. An immunogenic composition comprising the polypeptide of any one of claims 1-18 or the conjugate of any one of claims 20-22; and a carrier suitable for immunization purposes.

25. The immunogenic composition of claim 24, wherein the composition further comprises an adjuvant.

26. A method of producing an anti-MERTK antibody comprising (a) immunizing a non-human mammal with the polypeptide of any one of claims 1-18, the conjugate of any one of claims 20-22, or the immunogenic composition of claim 24 or 25; (b) immortalizing antibody producing cells from the non-human mammal to produce immortalized antibody-producing cells; (c) selecting an immortalized antibody-producing cell that secretes an antibody that immunospecifically binds MERTK; and (d) culturing the immortalized antibody-producing cell in a cell culture such that antibodies are produced.

27. The method of claim 26 wherein the step of immortalizing antibody-producing cells is carried out by a method comprising fusing the antibody-producing cells with myeloma cells to produce antibody-producing hybridomas.

28. The method of claim 26 or 27 which further comprises isolating the antibodies from the cell culture.

29. A method of identifying antibody sequences that encode an anti-MERTK antibody or antigen-binding fragment thereof comprising (a) immunizing a non-human mammal with the polypeptide of any one of claims 1-18, the conjugate of any one of claims 20-22, or the immunogenic composition of claim 24 or 25: (b) isolating antibody producing cells from the non-human mammal; (c) cloning antibody sequences of the antibody-producing cells to make a library of antibody sequences; (d) expressing antibody sequences in the library; and (e) selecting the antibody sequences that when expressed in the library produce an antibody or antigen-binding fragment thereof that immunospecifically binds to MERTK.

30. A method of screening candidate anti-MERTK antibodies or anti-MERTK antigen-binding antibody fragments comprising (a) assaying said antibodies or fragments for the ability to bind to the polypeptide of any one claims 1-18 or the conjugate of any one of claims 20-22; and (b) identifying one or more antibodies or fragments which immunospecifically bind to said polypeptide or conjugate.

31. The method of claim 30, wherein the assaying of antibodies or fragments for the ability to immunospecifically bind to said polypeptide or conjugate is done using an enzyme-linked immunosorbent assay (ELISA).

32. The method of claim 30 or 31, wherein the method further comprises a step of assaying one or more of the antibodies or fragments which immunospecifically bind to said polypeptide or conjugate for the ability to induce internalization of MERTK on human cells; and identifying one or more antibodies or fragments that induce internalization of MERTK on human cells.

33. The method of any one of claims 30-32, wherein the method further comprises a step of assaying one or more of the antibodies or fragments that bind to said polypeptide or conjugate for the ability to induce degradation of MERTK on human cells; and identifying one or more antibodies or fragments that induce degradation of MERTK on human cells.

34. A method of screening anti-MERTK antibodies or anti-MERTK antigen-binding fragments to identify an anti-MERTK antibody or anti-MERTK antigen-binding fragment that induces the internalization and/or degradation of human MERTK on human cells, the method comprising (a) assaying said antibodies or fragments for the ability to bind to the polypeptide of any one claims 1-18 or the conjugate of any one of claims 20-22; and (b) identifying one or more antibodies or fragments that immunospecifically bind to said polypeptide or conjugate, thereby identifying one or more antibodies or fragments that induce the internalization and/or degradation of human MERTK on human cells.

35. The method of claim 34 which further comprises assaying said one or more antibodies or fragments identified in step (b) for the ability to induce internalization and/or degradation of human MERTK on human cells; and identifying said one or more antibodies or fragments that induce internalization and/or degradation of human MERTK on human cells.

36. The method of claim 28 which further comprises purifying the isolated antibodies.

37. The method of claim 30 or 31 which further comprises purifying one or more of the antibodies or fragments that immunospecifically bind to said polypeptide or conjugate.

38. The method of claim 32, 34, or 35 which further comprises purifying one or more of the antibodies or fragments that induce internalization of MERTK on human cells.

39. The method of any one of claims 33-35 which further comprises purifying one or more of the antibodies or fragments that induce degradation of MERTK on human cells.

40. The method of any one of claims 26-29, wherein the mammal is a mouse.
